(19) United States
(12) Patent Application Publication
(10) Pub. No.: US 2022/0270713 A1
(43) Pub. Date: Aug. 25, 2022

(54) SYSTEMS AND METHODS FOR TARGETED INTENTIONAL MOLECULAR DESIGN

(71) Applicant: Phronesis Artificial Intelligence, Inc., Daphne, AL (US)

(72) Inventor: William Carl SPAGNOLI, Marina Del Rey, CA (US)

(21) Appl. No.: 17/584,073

(22) Filed: Jan. 25, 2022

Related U.S. Application Data
(60) Provisional application No. 63/151,377, filed on Feb. 19, 2021.

(51) Int. Cl. G16C 20/50; G16C 20/30

(52) U.S. Cl. CPC G16C 20/50; G16C 20/30

(57) ABSTRACT

Systems, devices, and methods for an iterative process for targeted intentional molecular design comprising: representing User Inputs in the form of a numeric matrix of one or more dimensions; using a model to predict a final metric or score assigned to a generated molecule upon completion for one or more actions if that action is used as the next design action taken in the molecule generation process; selecting one or more actions based on the predicted metric or scores; and generating one or more molecules based upon the selected actions.

### CROSS-REFERENCE TO RELATED APPLICATIONS

This application claims benefit of U.S. provisional patent application Ser. No. 63/151,377, filed Feb. 29, 2021, which is herein incorporated by reference.

### FIELD

Embodiments relate generally to molecular design, and more particularly to automated targeted intentional molecular design.

### BACKGROUND

Artificial intelligence (AI) techniques have been used to create improved pharmaceutical molecules. For example, minor improvements in drug design have resulted from the use of recurrent neural network language models, creating novel molecules based upon their similarity with known drugs and achieving a slightly targeted form of drug design. However, these molecules are derivatives of known molecules already in use and having proven efficacy. Additionally, the likelihood of pharmaceutical efficacy of the molecules generated in a recurrent neural network is not derivable from the recurrent neural network, as it generates only molecules having similar structures or sites thereon.

AI also has been employed to virtually screen the binding affinity of a protein in a molecule to a ligand, but to date cannot generate new molecules, but only predict the protein-ligand binding affinity for individual known molecular constructs or components thereof. Thus, a molecule having the protein for which the protein-ligand binding affinity has been determined can be selected, but the location of that protein may be on a portion of the molecule where the ligand cannot physically reach the protein, for example where the binding site location is recessed from the outer topography of the molecule and the size of the recess limits the ability of the protein and ligand to come close enough together to bind to one another.

These approaches to pharmaceutical molecule discovery suffer from a number of additional limitations preventing them from offering a full, effective solution to the problem of identifying new molecules that can serve as pharmaceuticals or pharmaceutical carriers. For example, in order to design effective pharmaceuticals, the drug attribute improvements must both be magnitudes greater than present approaches and multi-targeted, as a large amount of data concerning different molecular properties are needed for a new drug candidate to become FDA approved. Convolutional neural network computer vision models suffer from both the inability to achieve sufficient accuracy to provide comparable performance to pharmaceutical lab testing as a means of sorting which molecule candidates are likely to provide a beneficial effect, as well as the inability thereof to screen for any additional drug attributes beyond the single metric they are designed for. Due to these limitations, although prior AI applications have offered drug discovery assistance to pharmacologists, they fall far short of the human-expert-level performance required to properly mitigate the extensive timeline and resource scarcities hindering the medical industry.

### SUMMARY

Herein are provided methods and non-transitory computer media configured to generate molecules by repeatedly modifying a molecular structure of a molecule, and predicting, after at least one modification of the molecule to create an intermediate molecule structure prior to the generation of a final molecule structure, the properties of the molecule with respect to specified properties, and weightings of those properties, or of the molecule with respect to those properties.

In one aspect, this includes generating at least one of the chemical and physical structure of at least one molecule having a property by providing an initial molecule having at least one of a chemical structure and a physical structure, selecting at least a first attribute of the initial molecule relating to a first property thereof, evaluating the performance of the first molecule with respect to the first property thereof, modifying at least a portion of the at least one of a chemical structure and a physical structure of the initial molecule to form a first modified molecule, predicting the performance of the first modified molecule, upon further modification thereof, with respect to the performance of that first modified molecule with respect to the first property thereof, and based on the predicted performance, further modifying the first modified molecule.

In another aspect, a non-transitory computer-readable medium comprising instructions that, when executed by one or more processors of a computing system, cause the computing system to iteratively generate one or more molecular structures having desirable molecule properties is provided and includes representing user inputs in the form of a numeric matrix of one or more dimensions, predicting, using a model, a final metric or score assigned to a generated molecule upon completion for one or more actions, if that action were to be used as the next molecule design change action taken in the generation of one or more molecules, selecting one or more molecule design change actions based on the predicted metric or scores, and generating one or more molecules based upon the selected actions.

### BRIEF DESCRIPTION OF THE DRAWINGS

The components in the figures are not necessarily to scale, emphasis instead being placed upon illustrating the principals of the invention. Like reference numerals designate corresponding parts throughout the different views. Embodiments are illustrated by way of example and not limitation in the figures of the accompanying drawings, in which:

FIG. 1 depicts a top-level functional block diagram of a computing system environment;

FIG. 2 depicts components in communication with a processor of the computing system of FIG. 1;

FIG. 3 depicts a welcome screen of a computing device of the computing system of FIG. 1;

FIG. 4 depicts a settings page of the computing device of FIG. 3;

FIG. 5 depicts a receptor selection page of the computing device of FIG. 3;

FIG. 6 depicts a summary page of the computing device of FIG. 3;

FIG. 7 depicts a progress bar page of the computing device of FIG. 3;

FIG. 8 depicts a flow diagram of a system overview;

FIG. 9 depicts an output 5 folder of the computing device of FIG. 3;

FIG. 10 depicts an output file associated with the output folder of FIG. 9;

FIG. 11 depicts an output table associated with the output file of FIG. 10;

FIG. 12 depicts a molecular image;

FIG. 13 depicts an alternative molecular image;

FIG. 14 depicts a flow diagram of a molecular representation;

FIG. 15 depicts a flow diagram of an input preparation process;

FIG. 16 depicts a flow diagram of a prediction process performed by a molecular design component;

FIG. 17 depicts a schematic of one or more neural networks within the molecular design component of FIG. 16;

FIG. 18 depicts a flow diagram of the internal architecture of one of the neural networks of FIG. 17;

FIG. 19 depicts a flow diagram of additional internal componentry of one of the neural networks of FIG. 17;

FIG. 20 depicts a flow diagram of a Multi-Head Attention Layer of one of the neural networks of FIG. 17;

FIG. 21 depicts a flow diagram of an Encoder Block of one of the neural networks of FIG. 17;

FIG. 22 depicts a flow diagram of a Multiple-Transformer Neural Network;

FIG. 23 depicts a flow diagram of a molecule synthesis process;

FIG. 24 depicts a flow diagram of a molecular analyzer process;

FIG. 25 depicts a flow diagram of a system overview;

FIG. 26 depicts a block diagram of the system of FIG. 25;

FIG. 27 shows a high-level block diagram and process of a computing system for implementing an embodiment of the system and process;

FIG. 28 shows a block diagram and process of an exemplary system in which an embodiment may be implemented; and

FIG. 29 depicts a cloud computing environment for implementing an embodiment of the system and process disclosed herein.

### DETAILED DESCRIPTION

The described technology concerns one or more methods, systems, apparatuses, and mediums storing processor-executable process steps of automated targeted intentional molecular design allowing a user or users to design molecules of any desired traits and providing detailed metrics for the new molecules to the user or users. In one embodiment, an automated targeted intentional molecular design application may automatically provide organized, easy to understand, and sortable measurements of newly generated molecules, allowing the user to immediately view side-by-side comparisons of all relevant properties in new molecules. In one embodiment, the described technology utilizes reinforcement learning to allow a user or users of the automated targeted intentional molecular design application to design molecules of any desired traits and providing detailed metrics for the new molecules to the user or users.

AI also has been employed to virtually screen the binding affinity of a protein in a molecule to a ligand, but to date cannot generate new molecules, but only predict the protein-ligand binding affinity for individual known molecular constructs or components thereof. Thus, a molecule having the protein for which the protein-ligand binding affinity has been determined can be selected, but the location of that protein may be on a portion of the molecule where the ligand cannot physically reach the protein, for example where the binding site location is recessed from the outer topography of the molecule and the size of the recess limits the ability of the protein and ligand to come close enough together to bind to one another.

These approaches to pharmaceutical molecule discovery suffer from a number of additional limitations preventing them from offering a full, effective solution to the problem of identifying new molecules that can serve as pharmaceuticals or pharmaceutical carriers. For example, in order to design effective pharmaceuticals, the drug attribute improvements must both be magnitudes greater than present approaches and multi-targeted, as a large amount of data concerning different molecular properties are needed for a new drug candidate to become FDA approved. Convolutional neural network computer vision models suffer from both the inability to achieve sufficient accuracy to provide comparable performance to pharmaceutical lab testing as a means of sorting which molecule candidates are likely to provide a beneficial effect, as well as the inability thereof to screen for any additional drug attributes beyond the single metric they are designed for. Due to these limitations, although prior AI applications have offered drug discovery assistance to pharmacologists, they fall far short of the human-expert-level performance required to properly mitigate the extensive timeline and resource scarcities hindering the medical industry.

The techniques introduced below may be implemented by programmable circuitry programmed or configured by software and/or firmware, or entirely by special-purpose circuitry, or in a combination of such forms. Such special-purpose circuitry (if any) can be in the form of, for example, one or more application-specific integrated circuits (ASICs), programmable logic devices (PLDs), field-programmable gate arrays (FPGAs), etc.

The particular problems associated with molecular design, particularly for pharmaceutical molecules where the affinity of the molecule to bind to a receptor, for example the binding affinity between a protein on the molecule and a ligand in a virus, bacteria, or other harmful agent is important, but other factors, such as the molecular weight of the molecule and the solubility of the molecule in bodily fluids are also important, has rendered prior techniques for novel molecule generation less than adequate to provide an end user with candidate molecules likely to meet the needs of the user, for example a pharmaceutical company needing a molecule which can be used to treat a specific disease or infection. This is a result of the prior approaches able to consider only a single property of the molecule to be generated, or the molecules being derivative of known molecules having known efficacy, which limits the exploration into novel molecules. Herein, there is provided a methodology and media useful to weigh multiple desired properties of a molecule, iteratively generate intermediate molecules, and using each intermediate molecule, modify the intermediate molecule to generate a new intermediate molecule based on a prediction of how the modification will affect the final desired properties of the end or last molecule generated. This is herein provided using a neural network to generate changes in the intermediate molecules and predict how the modification will affect the desired properties of the final molecule, and a molecular analyzer which generates a scoring of the molecule, based on the weights assigned to different properties thereof and the usefulness of the molecule based on those properties.

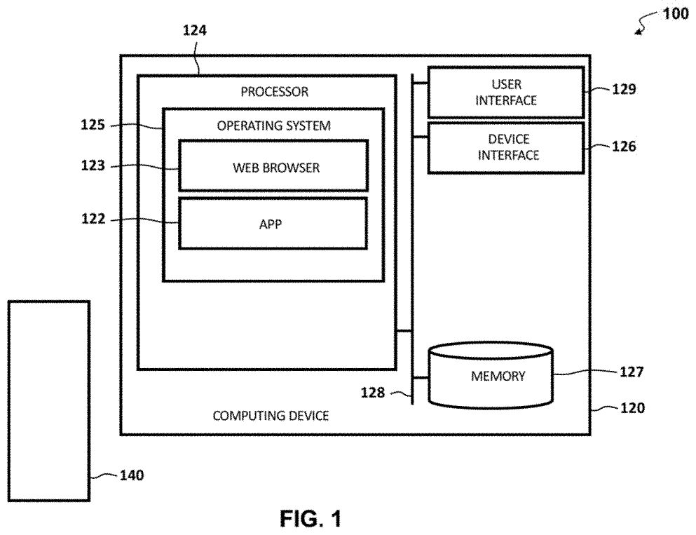

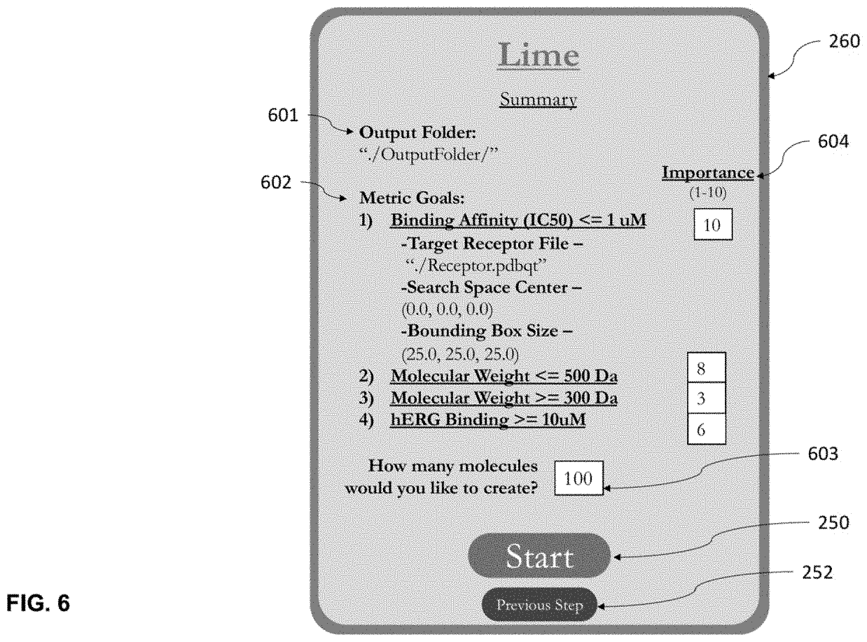

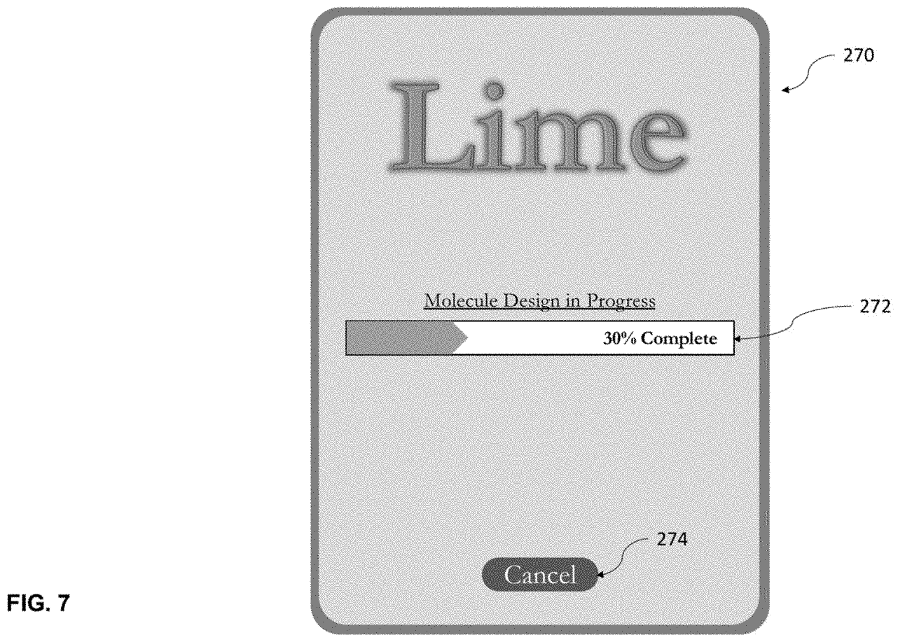

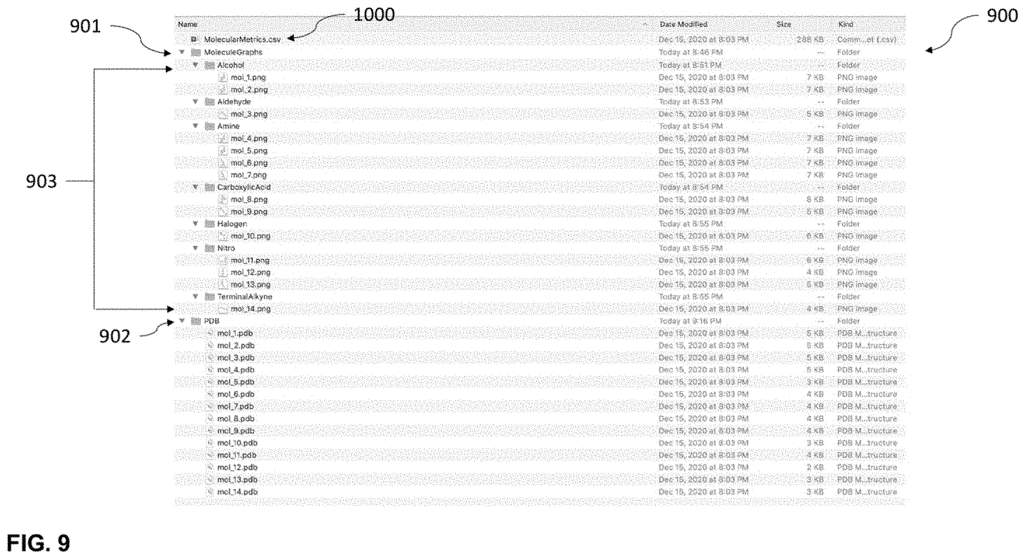

FIGS. 1-25 and the following discussion provide a brief, general description of a suitable computing environment in which aspects of the described technology may be implemented. Although not required, aspects of the technology may be described herein in the general context of computer-executable instructions, such as routines executed by a general- or special-purpose data processing device (e.g., a server or client computer). Aspects of the technology described herein may be stored or distributed on tangible computer-readable media, including magnetically or optically readable computer discs, hard-wired or preprogrammed chips (e.g., EEPROM semiconductor chips), nanotechnology memory, biological memory, or other data storage media. Alternatively, computer-implemented instructions, data structures, screen displays, and other data related to the technology may be distributed over the Internet or over other networks (including wireless networks) on a propagated signal on a propagation medium (e.g., an electromagnetic wave, a sound wave, etc.) over a period of time. In some implementations, the data may be provided on any analog or digital network (e.g., packet-switched, circuit-switched, or other scheme).

The described technology may also be practiced in distributed computing environments where tasks or modules are performed by remote processing devices, which are linked through a communications network, such as a Local Area Network (“LAN”), Wide Area Network (“WAN”), or the Internet. In a distributed computing environment, program modules or subroutines may be located in both local and remote memory storage devices. Those skilled in the relevant art will recognize that portions of the described technology may reside on a server computer, while corresponding portions may reside on a client computer (e.g., PC, mobile computer, tablet, or smartphone). Data structures and transmission of data particular to aspects of the technology are also encompassed within the scope of the described technology.

Present embodiments provide for automated targeted intentional molecular design wherein a user may be presented with newly-designed molecules that are automatically organized, easy to understand, and sortable measurements, allowing the user to immediately view side-by-side comparisons of relevant properties in the newly-designed molecules. In one embodiment, “Fully Automated Intentional Molecular Design” (FAIMD) may execute a program to prepare an input representation of user inputs and the current state, i.e., the current physical or chemical, or both, structure, of the molecule being designed and provide said input representation to a model to predict the final “Reward Score” to be received by a final, fully designed molecule if a specific molecular design action is selected, for each possible next molecular design action that may be selected. More specifically, the system 100 may create a vector of conditional predictions —one prediction for each possible action it may choose. In one embodiment, each number within this vector may be a predicted final score contingent on the respective action being selected. For example, imagine someone going to a job interview and at the end of the interview that person either gets the job [1] or does not [0]. When the person first walks into the interview, the person may predict that if they start off with an inappropriate joke, they will not got the job (expected final reward of 0). The person also predicts that if they present the hirer with their résumé and they comport themselves professionally, the person will end up getting the job (expected final reward of 1). Therefore, the person chooses to start with the latter because it has a greater expected final reward. In the same manner, as the person is sitting down talking to the interviewer, the person then predicts that if they tell the interviewer something relatable, the person will get the job (expected final reward of 1), and the person further predicts that if they tell the interviewer they have no weaknesses, they will not get hired (expected final reward of 0); therefore, the person choose to be honest and relatable to maximize their expected final reward. Throughout the entire interview, every action the person makes is based on predicting how the interview would end conditional on taking the various available choices, and the person may use that prediction to consistently select the actions that maximize their expected final reward at the end of the interview. In this same manner, 1 the Neural Network component of the system 100 may predict “If I add a Hydrogen atom next, the final molecule will probably end up with a score of 9 when I finish, but if I add a Carbon atom next, the final molecule will probably end up with a score of 8 when I finish”, and so on. In one embodiment, the expected final reward numbers may be used to select the next action. In one embodiment, the actions may be sampled stochastically.

In one embodiment, the “Reward Score” may be a total score calculated by a Molecular Analyzer Component, such as Molecular Analyzer Component (172, 2000) described below, representing the overall quality of the newly designed final molecule with respect to each of the target molecular metric goals. The system may then select a next molecular design action, update the input representation to reflect that molecular design action being taken, and continuously repeat this process until an “End” action is selected by a user. Once the end action is selected by the user, the newly designed, final molecule is given to the Molecular Analyzer Component, all molecule output files are saved to an Output Folder, such as Output Folder (900) described below, and this process may be repeated a certain number of times specified by a User Input in a setting, such as in a “How many molecules would you like to create?” setting (603) described below.

The targeted intentional molecular design system provides an easy-to-use user interface, which allows artificial intelligence (AI) molecular design to be used by researchers in any industry, not only limited to software developers. As such, the targeted molecular design system may be accessible to anyone who needs it, regardless of technological expertise.

The robust targeting algorithm of the targeted molecular design system provides enhanced control over molecular design. For example, when used for drug discovery, the user may want a molecule that not only has a sufficient binding affinity with a target pathogen but also can be administered orally and is simple to synthesize. Alternatively, a non-medical user may wish to target specific pH levels or molecular weight. The targeted molecular design system provides the user with a robust ability to choose a variety of molecular qualities that the user may wish to create. In other embodiments, the targeted molecular design system may provide for new targeting functions to be easily added by a user.

The targeted molecular design system addresses a variety of problems across different fields that require an understanding of a diverse collection of fields. For example, the targeted molecular design system not only provides for optimizing binding affinity, but also has the domain knowledge of the pharmaceutical industry, drug discovery process, and FDA regulations/barriers to drug approval. Therefore, the targeted molecular design system may understand the need and required attributes for simultaneously targeting other ideal drug qualities. In the same manner, the targeting of desired attributes for industrial/chemical compounds requires additional domain knowledge of chemistry and material science, which the targeted molecular design system possesses.

It is understood that while molecules with strong-binding affinity to the target receptor are a good start for discovering a candidate drug, strong-binding affinity is one of many necessary molecular qualities for effective drugs.

For example, Remdisivir® has shown great potential as a candidate drug for COVID-19 throughout the current global pandemic due to its binding affinity to the virus' ACE2 receptor but presents challenges in the production of a global supply due to the complexity required to synthesize the molecule. Additionally, high-quality drug candidates must not have adverse interactions with other drugs and/or the human body, be able to permeate through the necessary membranes for absorption, preferably be soluble enough to be orally administered (for patient acceptance) and meet many more requirements. The present embodiments provide for a system that may not only target strong-binding affinity molecules and other desired traits but also for information regarding adverse interactions with other drugs and other pertinent information, such as FDA requirements.

Additionally, a user-friendly interface of the targeted intentional molecular design system provides for easy operation for newly-designed molecules with any desired traits for non-tech savvy users, allowing for wide-spread adoption across industries.

The targeted intentional molecular design system provides enhanced efficiency in the molecular design process, an essential process for a wide range of fields, including, but not limited to, drug discovery, industrial material design, chemical innovation, and many more fields. This inefficiency in the molecular design process is due to the vast complexity of molecule design and molecular interactions. There are estimated to be between 1060 and 1080 unique molecules currently in existence with only an estimated 60 Million currently known, documented molecules. The targeted molecular design system may efficiently probe the vast universe of possible molecules, greatly speeding up the design and discovery of new molecules with desired traits. For example, in drug discovery, narrowing down to the top 250 candidate drugs to take to clinical trials typically may take anywhere from 4-7 years, requiring hundreds of millions of dollars and entire teams of experts. The targeted molecular design system may remove these barriers and provides all forms of molecular design, from drug discovery to chemical compound design, in a quick and easy interface with little to no experience required.

The present embodiments not only assist in the field of drug discovery, but they also provide algorithms able to solve many of humanity's needs for new molecules. For example, society needs a solution that will design a stronger new metal alloy able to save a child in a car crash, a new chemical to fill exit signs to avoid radiation exposure, and countless other molecules that offer the potential to save lives.

The present embodiments provide for a simple, user-friendly system that makes the target molecular design state-of-the-art technology accessible to everyone, regardless of experience. FIG. 1 illustrates an example of a top-level functional block diagram of a computing system embodiment (100). The example operating environment is shown with a server computer (140) and a computing device (120) comprising a processor (124), such as a central processing unit (CPU) or a graphics processing unit (GPU), addressable memory (127), an external device interface (126), e.g., an optional universal serial bus port and related processing, and/or an Ethernet port and related processing, and an optional user interface (129), e.g., an array of status lights and one or more toggle switches, and/or a display, and/or a keyboard and/or a pointer/mouse system and/or a touch screen. Optionally, the addressable memory may include any type of computer-readable media that can store data accessible by the computing device (120), such as magnetic hard and floppy disk drives, optical disk drives, magnetic cassettes, tape drives, flash memory cards, digital video disks (DVDs), Bernoulli cartridges, RAMs, ROMs, smart cards, etc. Indeed, any medium for storing or transmitting computer-readable instructions and data may be employed, including a connection port to or node on a network, such as a LAN, WAN, or the Internet. These elements may be in communication with one another via a data bus (128). In some embodiments, via an operating system (125) such as one supporting a web browser (123) and applications (122), the processor (124) may be configured to execute steps of a process establishing a communication channel and processing according to the embodiments described above. In one embodiment, an application (122) is a targeted molecular design application as described below.

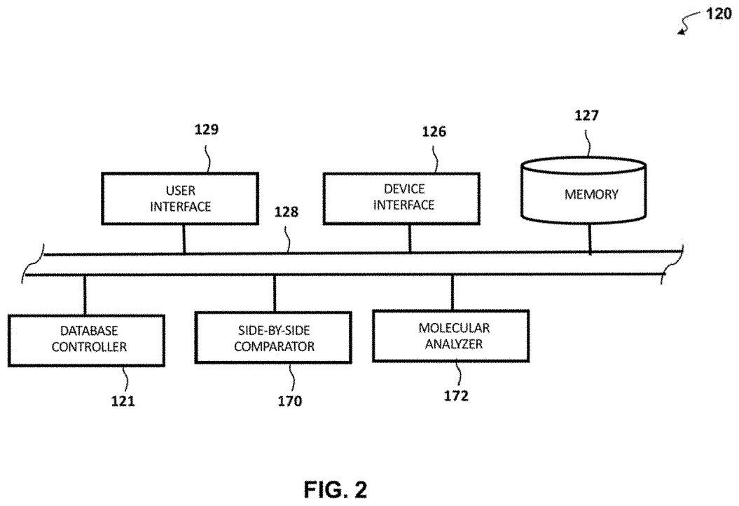

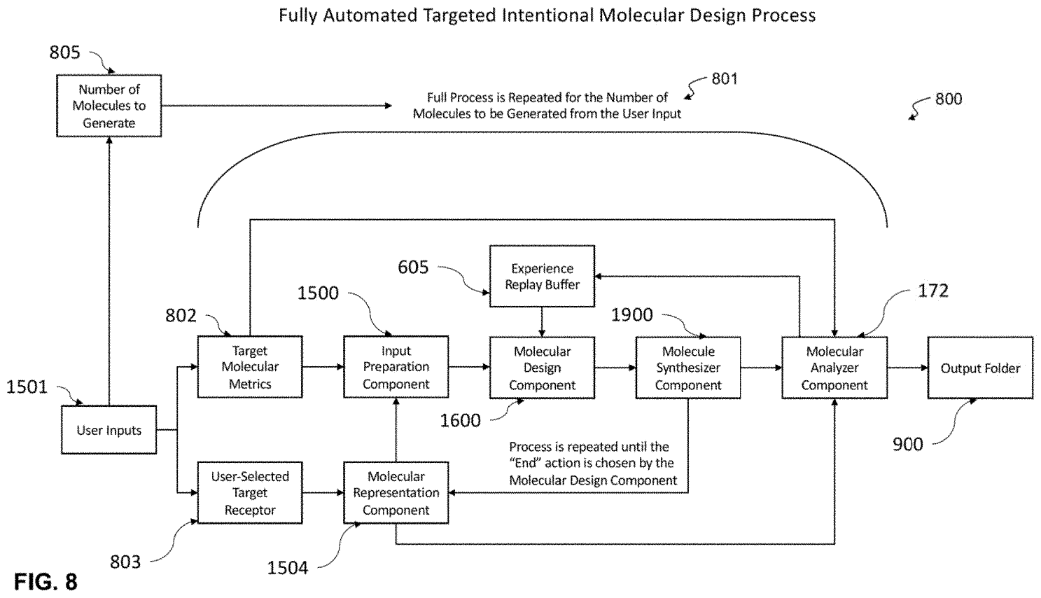

With respect to FIG. 2, components associated with or in communication with the processor (124) are shown. A database controller (121) may be in communication with the processor (124), for example, via the data bus (128). In one embodiment, the database controller (121) may receive and store data, such as data from various industries (e.g., pharmaceutical industry, chemical industry, FDA, etc.) as well as a library of different molecules from at least one database, such as a database associated with the server computer (140) in FIG. 1, and load said data into, for example, a cross-platform database 5 program. More specifically, protein receptor files, Experience Replay Buffer data, previous experiment history, and other past files may be uploaded by a user. A user may launch the targeted intentional molecular design application (e.g., application 122) to interact with the program at the user interface (129). The application (122) may then use access this database at any point to use or store any files used within molecular design process, such as the Fully Automated Targeted Intentional Molecule Design Process (800) described in FIG. 8, or in any other processes facilitated by the application (122). The system (100) may provide for targeted molecular design allowing a user to design molecules of any desired traits, and automatically providing detailed metrics for the new molecules to the user or users. In one embodiment, the side-by-side comparator component (170) may automatically present organized, easy to understand, and sortable measurements of all newly generated molecules to the user at the user interface (129), allowing the user to immediately view side-by-side comparisons of all relevant properties in new molecules.

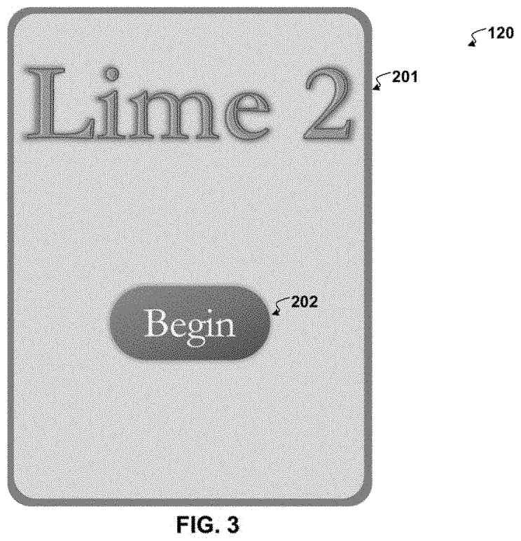

With respect to FIG. 3, in one embodiment, the user may be presented with a welcome screen (201) with a “begin” toggle button (202) at the computing device (120). In one embodiment, the user may be presented with the welcome screen upon launching the target molecular design application.

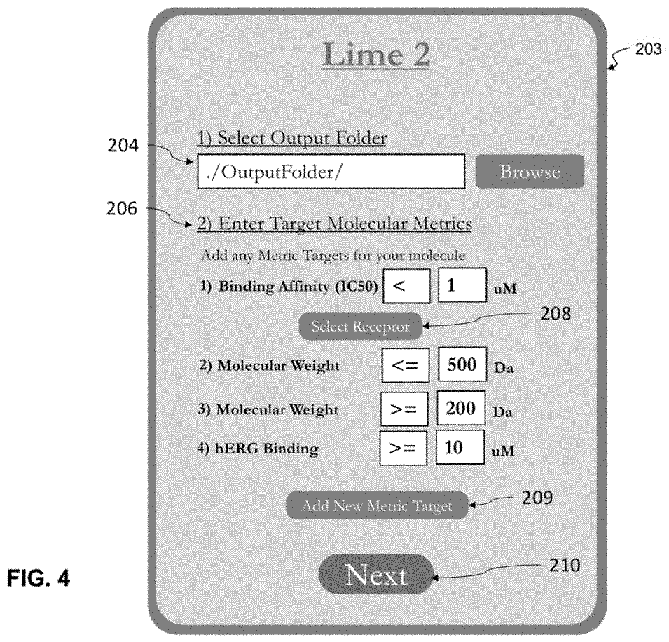

Once the user selects the “Begin” button (202), the user is taken to a Settings Page (203), as shown in FIG. 4. The settings page displays the required settings at the user interface that the user must complete in order to run the targeted intentional molecular design application. In one embodiment, a first setting (204) is selected by the user at the user interface (129) to choose an output folder on a computing device, such as computing device (120) where the application (122) saves all molecule information and other files it creates. For example, an Output Folder may contain molecular metrics output files, such as tables containing molecules and their respective molecular metrics, 2-D molecular images, and 3-D molecular images. A “Model Checkpoints” folder may save at least one Hierarchical Data Format version 5 (HDF5) file, which include the system's Neural Network Component 5 training checkpoints. A “Model Training” folder contain data received by one or more Experience Replay Buffers, such as numerical representations of final prepared inputs, selected actions, numerical vector of Final Molecular Measurement Scores, total final reward scores, and other data used to train the Neural Network Component. This data received by the Experience Replay Buffers may be stored in Comma Separated Value (CSV) format, pickle format, or other file formats capable of storing the data within the Experience Replay Buffers. In another embodiment, additional files may be included. In yet another embodiment, the system may have a user-friendly, icon-based file organization.

A second setting (206) allows to the user at the user interface (129) to choose one or many “Target Molecular Metrics” selecting the “Add New Metric Target” Button (209) and inputting the Target Molecular Metric, which, in one embodiment, may be represented in the User Inputs as concatenated vectors of the numeric input target, numeric Importance Score, a one-hot-encoded vector representing the metric, and a one-hot-encoded vector representing the comparison operator, combined into a numeric matrix array representation.

In one embodiment, the Target Molecular Metrics selected by the user may be received at the Molecular Analyzer Component (172) and the Input Preparation Component, indicating the molecular qualities that the Molecular Design Component may design molecules to achieve. The Input Preparation Component appends a numeric representation of the User Inputs to the final prepared input into the model to provide design instructions to the Molecular Design Component. The Molecular Analyzer Component (172) uses the User Inputs to analyze newly designed molecules by calculating a numerical vector of Final Molecular Measurement Scores of each separate metric goal and calculate a total final reward score representing the molecule's total overall performance across all target metrics goals. Both the numerical vector of all Final Molecular Measurement Scores and total final reward score may be received by one or many Experience Replay Buffers to provide training data to further improve the performance of the Neural Network Component through training the model via back-propagation or other optimization strategy.

For example, if a user wishes to design a cure for a specific disease, as demonstrated in FIG. 4, the user may input the target metric goal of “Binding Affinity (IC50)<1 uM” to ensure inhibition of the target receptor, then input the target metric goals of “Molecular Weight <=500 Da” and “Molecular Weight >=200 Da” (A required test within the Rapid Elimination of Swill (REOS) Drug Filter), and “hERG Binding >=10 uM” in order to avoid the design of molecules with a high probability of resulting in side effects causing heart arrhythmias. When designing molecules for other purposes, or with additional target metric goals, the user is able to select any combination and number of molecular metrics to be included as the selected target metric goals. Additionally, the user can set the target metric goals to have different, or the same, weight or value.

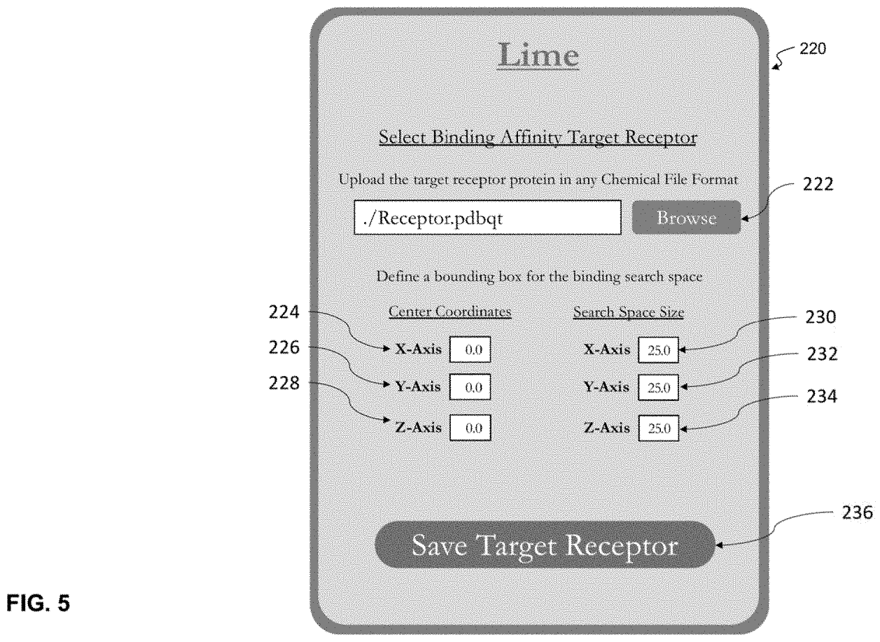

In one embodiment, when the user selects “Binding Affinity (IC50)” as the Target Molecular Metric, the user must select a “Select Receptor” button (208) which opens a Receptor Selection page (220) shown in FIG. 5. At the Receptor Selection page (220), the user may upload, using a Browse Button (222), a protein structure file of the receptor that they wish to target in the form of a PDBQT file, mol2 file, or another chemical file format. In another embodiment, the target receptor may be provided in the form of a 3-D matrix, Simplified Molecular-Input Line-Entry System (SMILE) format, or other chemical representation format. For example, if a user wanted to design a drug to combat COVID-19, the user may select to upload a “.PDBQT” file of the Spike Protein, which is used by the virus to enter human cells. This protein structure file can then be used by the Input Preparation Component to provide a Molecular Design Component, such as Molecular Design Component (1600) described in further detail in FIG. 8 with a numerical representation of the molecular design instructions. Additionally, this protein structure file can then be used by the Molecular Analyzer Component (172) to score all newly designed molecules on the molecule's ability to inhibit the spike receptor based on the respective molecule's measured Half-Maximal Inhibitory Concentration (IC50) against the target receptor. As such, the molecular analyzer component (172) measures how well each molecule would be able to prevent COVID-19 from entering human cells.

In one embodiment, once the user has uploaded the receptor file, the user needs to define a bounding box, which dictates which part of the receptor will be analyzed when measuring binding affinity. Here, for example, the user uploads a location for the bounding box, as well as the size of the sides of, or the volume of, the bounding box. In one embodiment, the user may enter numerical values for center coordinates to center on the receptor. The center coordinates may be x, y, and z coordinate values entered at an X-axis box (224), Y-axis box (226), and Z-axis box (228) coordinate boxes, respectively. In one embodiment, the boxes (224, 226, and 228) may have a default value of 0.0. In one embodiment, the user enters numerical values for the search space size of the receptor. The search space size may be x, y, and z coordinate values entered at an X-axis box (230), Y-axis box (232), and Z-axis box (234) coordinate boxes, respectively. In one embodiment, the boxes (230, 232, and 234) may have a default value of 25.0 Angstrom units. In another embodiment, the user will not be required to define a bounding box in Angstrom units. Once the user has entered all of the receptor information, the user may press a “Save Target Receptor” Button (236) which will save the receptor information and return the user to the Previous Settings Page (203).

Once the user has finished inputting the desired settings, the user may press a “Next” button (210) to be taken to a Summary Screen (260) displayed at the User Interface (129). In one embodiment, the Summary Screen (260) provides an Output Folder list (601) and a target Metric Goals list (602) of all the settings chosen by the user for confirmation. In one embodiment, users may be able to assign Importance Scores (604) to each molecular metric target to allow weighted targeting in which the Molecular Design Component (1600) prioritizes the performance of target molecular metrics according to the respective Importance Score (604) when creating a list of vectors, such as Expected Final Score Output Action Vectors. Different metric targets can be assigned different, or the same, Importance Score

As a final setting, the user may input the number of target molecules to be generated by inputting the number into a “How many molecules would you like to create?” Button (603), and a Fully Automated Targeted Intentional Molecule Design Process (e.g., Fully Automated Targeted Intentional Molecule Design Process (800) described in FIG. 8 below) may be automatically executed by the targeted intentional molecular design application (122) iteratively for the number of times specified by this input in order to generate the desired number of new molecules. The user may go back to change their settings using the “Previous Step” button (252), or if the user does not wish to make changes, they may click the “Start” button (250) to begin the Fully Automated Targeted Intentional Molecule Design Process.

In one embodiment, upon clicking the Start button (252), the targeted intentional molecule design process may begin automatically, and the user is directed to a Progress Bar Screen (270), as shown in FIG. 8. This Progress Bar Screen (270) may include a progress bar (272), where the user may view the percentage of the total process completed by the targeted intentional molecular design application (122). The user may cancel the process at any time by selecting a cancel button (274).

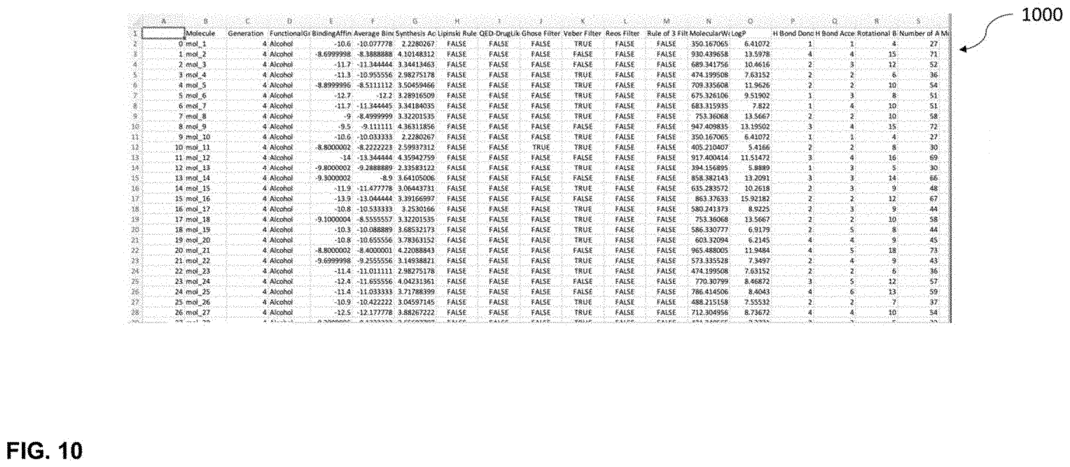

With respect to FIG. 8, a flow chart (800) depicts an iterative process for the Fully Automated Targeted Intentional Molecule Design Process (800). First, any Target Receptor(s) (803) provided for any target molecular metrics selected in a User Inputs numeric representation (1501), if any were selected target molecular metrics required a target receptor, are provided to a Molecular Representation Component (1504), converted into a numeric matrix representation (1505), and then provided to both an Input Preparation Component (1500) for design instructions and to the Molecule Analyzer Component (172) for scoring. Simultaneously, Target Molecular Metrics (802) may be provided to both the Input Preparation Component (1500) for design instructions and to the Molecule Analyzer Component (172) for scoring. The Input Preparation Component (1500) then provides a final prepared input vector (1509) to a Molecular Design Component (1600). The Molecular Design Component (1600) then provides a Predicted Final Reward Vector (1603) (explained in further detail below) to a Molecule Synthesizer Component (1900). The Molecule Synthesizer Component (1900) selects the next action in the molecular design process based upon the Predicted Final Reward Vector (1603), applies the respective molecule design action on the partially designed molecule and provides it to the Molecular Representation Component (1504), and this process is repeated iteratively. Once the Molecule Synthesizer Component (1900) selects the “End” action as the next action, this iterative loop completes, and the final, newly designed molecule is provided to the Molecule Analyzer Component (172) for scoring. The “End” action occurs when the system predicts that the next action should be the end action, thus completing the design of that specific molecule. The Molecule Analyzer Component (172) measures molecules across a large variety of molecular attributes and calculates all reward scores (e.g., reward scores (603)). The Molecule Analyzer Component (172) provides all data, such as numerical representations of final prepared inputs (1509 reward), selected actions 5 (1903), numerical vector of Final Molecular Measurement Scores (2002), total final reward scores (603), and other data used to train a Neural Network Component (1602) to an Experience Replay Buffer (605) to provide training data to further improve the performance of the Neural Network Component (1602) through training the model via back-propagation or other optimization strategy. Simultaneously, the Molecule Analyzer Component (172) saves all molecule measurement output files to an Output Folder (900) shown in FIG. 10, and then this process is iteratively executed for the number of times specified as the Number of Molecules to Generate (805) specified in the User Inputs (1501).

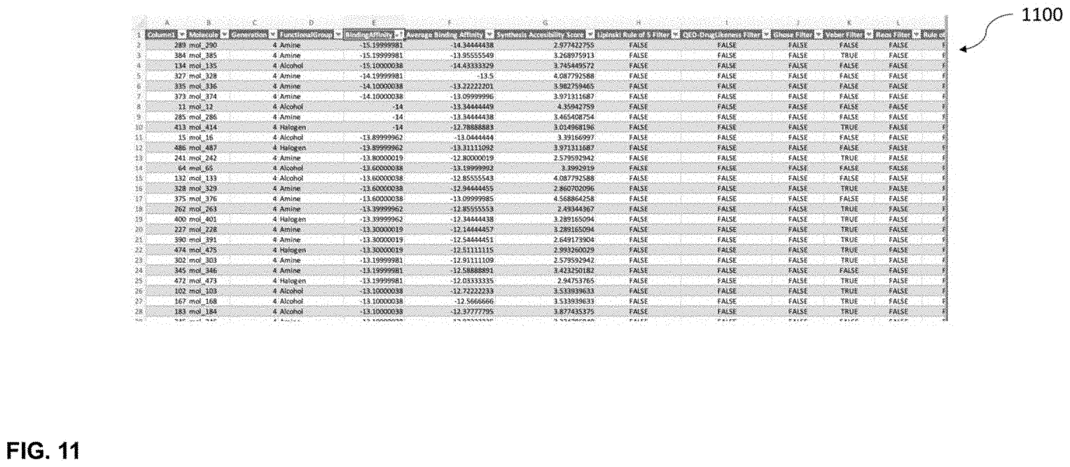

In one embodiment, the Output Folder (900) is selected by the user on the Required Setting Screen (203). A file, such as a “.csv” file (1000) shown in FIG. 10, with all of the molecules and their corresponding properties is saved to the Output Folder (900). The file (1000) may be easily converted into a sortable, filterable table (1100), such as an Excel file shown in FIG. 11. The table (1100) may allow users to quickly and easily view and compare top scoring molecules.

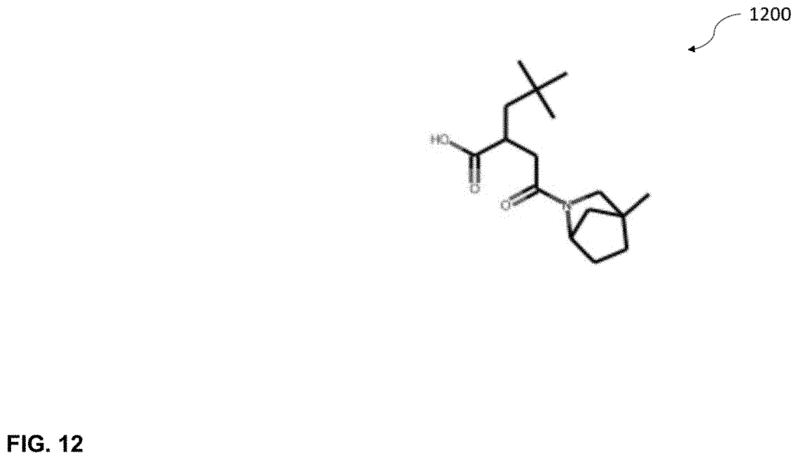

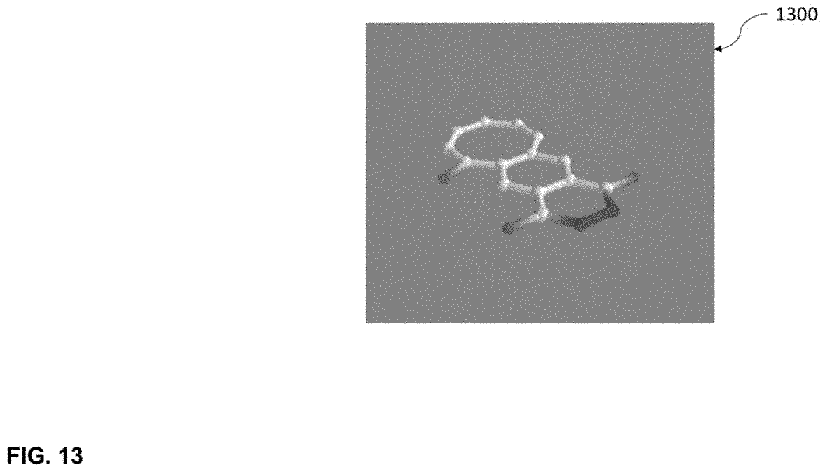

Along with the “.csv” file (1000) shown within the Output Folder (900), the Output Folder (900) contains additional subfolders: a “MoleculeGraphs” folder (901) and a “PDB” folder (902). The “MoleculeGraphs” folder (901) may contain molecular graph images, such as molecular image (1200) shown in FIG. 12 and a molecular image (1300), shown in FIG. 13. The molecular images (1200) provide 2-D representations of each molecule, conveniently stored into subfolders organized by molecular functional group (903) within the “MolecularGraphs” Folder (901), and the molecular images (1300) provide 3-D representations of each molecule stored in the “PDB” Folder (902). In another embodiment, the Output Folder (900) may contain additional files providing information regarding the molecules and may be organized into folders in an alternative pattern.

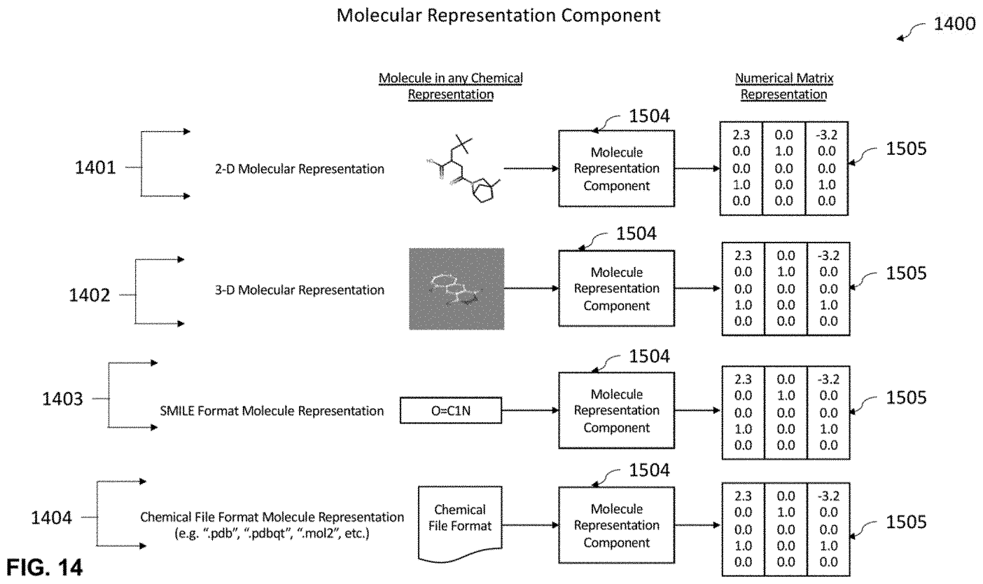

With respect to FIG. 14, a flow diagram (1400) of the function of the Molecular Representation Component (1504) described in FIG. 8. In some embodiments, the Molecular Representation Component (1504) may receive a molecule in a 2-D representation (1401), a 3-D representation (1402), a SMILE Format (1403), a Chemical File Format (1404), or other molecular representation. The molecular representation may then be tokenized by the Molecular Representation Component (1504) to split the input molecular representation into a vector of individual pieces. Tokenizing the input molecular representation breaks the representation into individual pieces of molecular information such as atom types and coordinates, molecular bonds, and other molecular properties described within the input molecular representation. The tokenization process may vary depending on the format of the input molecular representation.

For example, if the input molecular representation is a 3-D image representation (1402), the 3-D pixel location coordinates and types of different molecular attributes such as atoms and bonds may each be represented separately in the vector of individual pieces. Similarly, if the input molecular representation is a string of text in SMILE Format (1403), the SMILE format text string may be broken into individual linguistic units, or more specifically, the SMILE format representation molecules may be split into each individual character. Input molecular representations in the Chemical File Format (1404) may be broken into individual lines within the file, which each represent different molecular attributes defining 3-D structural information such as atomic types, coordinates, and bonds similar to the 3-D data extracted from 3-D image representations. These molecular attributes may be automatically extracted from the Chemical File using text splitting functions commonly provided automatically by programming languages, by using custom data extract functions, or by using third-party software. In another embodiment, input molecular representations may be automatically converted to different molecular representations using third-party software (e.g. RDKit) or other molecule format conversion functions. In one embodiment, the Molecular Representation Component (1504) is able to take any molecular representation as input and create the Numerical Matrix Representation Matrix regardless of the input molecular representation. After the atomic properties have been extracted from the input molecular representation and split into the vector of individual pieces, each individual piece may be one-hot-encoded into a binary vector representation and concatenated with a value of 0 for categorical pieces or the respective numerical value if the respective piece is a number, resulting in a Numerical Matrix Representation (1505) of each molecule. In another embodiment, numerical matrix representations may be formatted differently.

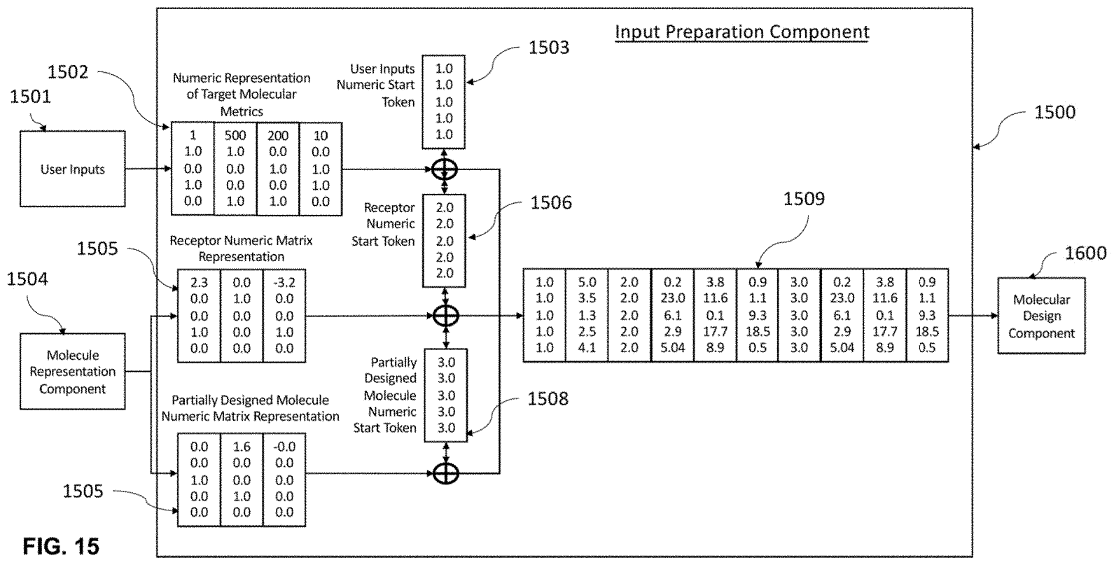

With respect to FIG. 15, a flow diagram of a process 1400 of the Input Preparation Component (1500) is shown. The Input Preparation Component (1500) initially receives the numerical matrix representations (1505) for all receptors, if any, required for the target molecular metrics and the partially designed molecule. Simultaneously, the Input Preparation Component (1500) receives a numeric representation of target molecular metrics (1502). The Input Preparation Component (1500) then concatenates a User Inputs Numeric Start Token (1503) with the User Input Numerical Matrix Representation (1502), a Receptor Numeric Start Token (1506) with any receptor numeric matrix representations, a Partially Designed Molecule Numeric Start Token with the partially designed molecule numerical matrix representation, then concatenates all of these matrices into a final, prepared input numeric matrix (1509). This final, prepared input numeric matrix (1509) is then provided to the Molecular Design Component (1600) to begin the next design step of the Fully Automated Targeted Intentional Molecule Design Process (800).

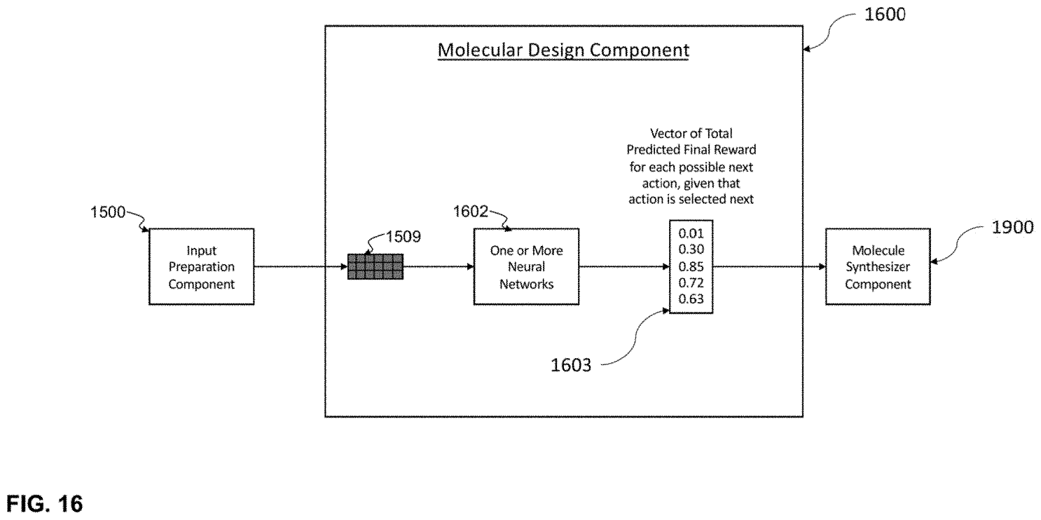

With respect to FIG. 16, a flow diagram of the Molecular Design Component (1600) function is shown. The Molecular Design Component (1600) receives the numeric input matrix (1509) from the Input Preparation Component (1500) and passes it through One or More Neural Networks (1602) to predict the final score given by the Molecule Analyzer Component (172) that would be achieved if a specific molecular design action was to be selected and the One or More Neural Networks (1602) maintain the current decision-making policies until the final molecule is fully designed. Furthermore, the one or more Neural Networks may output an Expected Final Score Vector (1603) containing one number for each possible next molecular design action which may be chosen, each representing the predicted final molecule score which would be achieved if the respective molecular design action is selected as the next molecular design action to be taken. As this is a relatively complex mathematical concept of Artificial Intelligence, a simple metaphor to better explain this process step is to picture a football game, and a viewer is asked to predict the final score of the game that the home team will receive. Rather than only predicting one final score, viewer may predict that if the quarterback's next action is to throw a touchdown pass on the next play, the home team will likely receive a final score of 21, but if his next action is to throw an interception, the home team will likely receive a final score of 14. In the same way, the One or More Neural Networks (1602) predict what the final score received by the Molecule Analyzer Component (172) would be in the event of each next molecular design action which may be selected. This Expected Final Score Vector (1603) output is then received by the Molecule Synthesizer Component (1900).

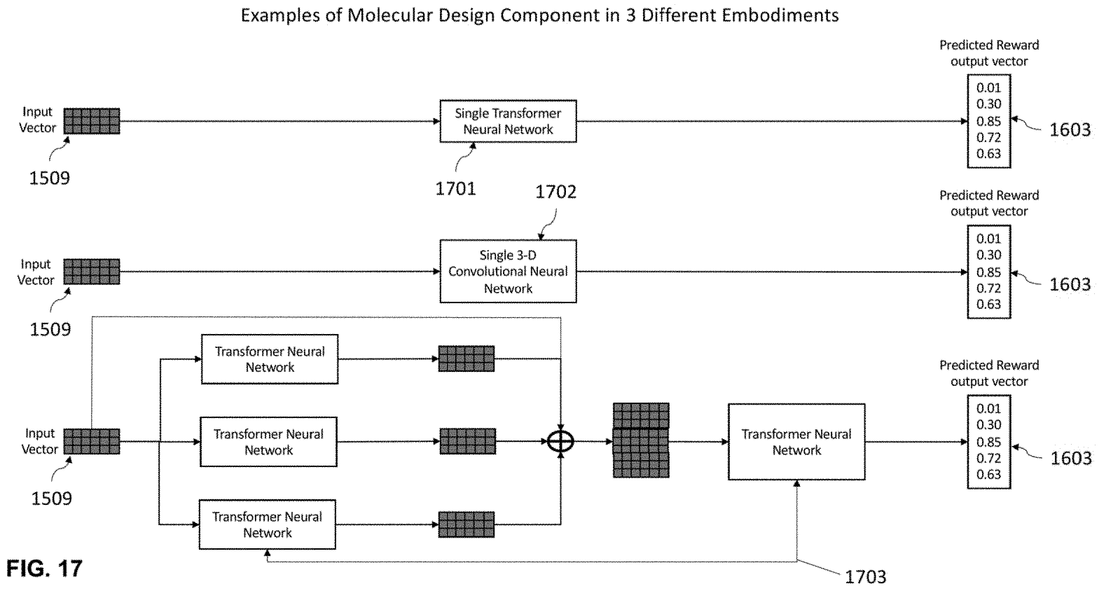

With respect to FIG. 17, flow diagrams of the One or More Neural Networks Components (1602) used in different embodiments are shown. In other embodiments, the One or More Neural Networks Component (1602) may be constructed using a different model architecture consisting of at least one Neural Network to compute the Expected Final Score Vector (1603) using the Prepared Input Matrix (1509). The flow diagrams depicted in FIG. 17 are only meant to be examples to demonstrate the necessity of utilizing different designs of the One or More Neural Networks Component (1602) required to allow this technology to become widely accessible and allow all of humanity to reap the benefits the technology may provide, regardless of economic status. For example, if an impoverished nation is plagues by a rare, novel disease, the country's pharmacologists may not have access to adequate computing resources required by a large Neural Network, leaving them unable to reap the benefits of fully automated targeted intentional molecular design, delaying their ability to discovery a cure over a decade and resulting in countless deaths that could have been avoided. This tragic disaster may be avoided through the creation of varying embodiments utilizing different designs of the One or More Neural Network Component (1602). For this scenario, the use of a smaller Neural Network such as the 3-D Convolutional Neural Network (1702) may allow fully automated targeted intentional molecular design to operate on a computationally weak device, such as a smartphone or laptop.

While such designs may come at the expense of targeting precision, they may still reduce the drug discovery timeline by many years, saving lives in resource-sparse settings. Alternatively, with sufficient computation resources, a large ensemble of many transformer Neural Networks as (1703) may achieve significantly higher targeting precision. Given vast amounts of both data and computational resources, a much larger, single transformer Neural Network as (1701) is likely to achieve even further improvements in targeting precision. The depicted ensemble of many transformer Neural Networks (1703) would operate in a mathematically similarly manner to the depicted large, single-transformer Neural Network (1701) due to the ensemble design utilizing an attention mechanism on an input (1804) consisting of both the Final Prepared Input Vector (1509) and concatenated outputs (1803) from other transformer models, but is able to utilize transfer learning and incremental learning strategies (described in further detail below) to reduce computational costs. The single large transformer Neural Network (1701) may naturally allocate parameters to compute similar molecular attributes as are calculated by each transformer within the ensemble of transformer Neural Networks (1703) while having a much more robust capability to understand the intercorrelated relationships between the metrics. However, given the complexity of the problem to be solved, a single large transformer Neural Network (1701) would likely require one of the largest Neural Networks created in the industry so far, requiring vast amounts of data and computational resources. Additionally, with the rapid pace of innovation within the Artificial Intelligence industry, new algorithmic discoveries to improve Neural Network performance are published nearly on a daily basis. Through the continuous release of new embodiments utilizing cutting-edge algorithms to enhance the performance of the One or More Neural Networks Component (1602), the life-saving societal benefits of this technology can be maximized as newly discovered algorithmic improvements can be applied to the One or More Neural Networks Components within weeks of discovery, providing consistent, widespread access to the power of the most cutting edge algorithms the field of Artificial Intelligence has to offer.

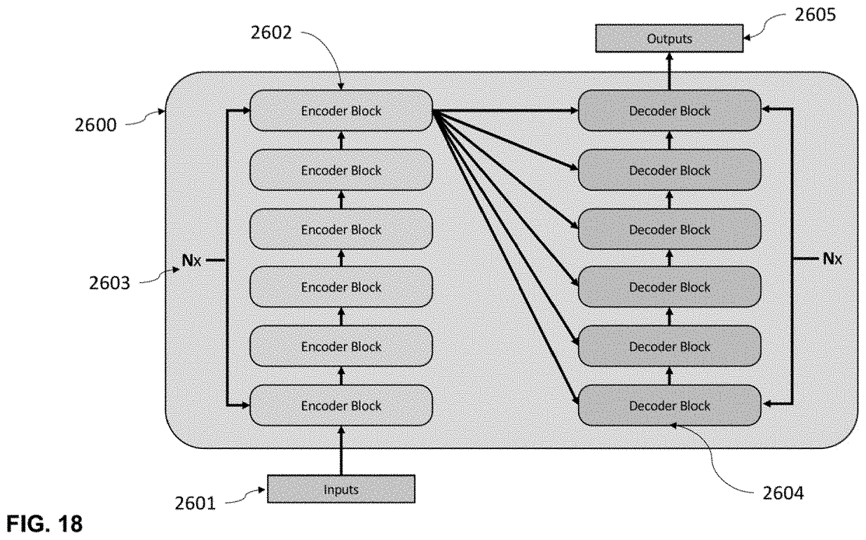

With respect to FIG. 18, a flow diagram of a Transformer Neural Network (2600) within the large ensemble of many Transformer Neural Networks (1703) depicted in FIG. 17 is shown.

In one embodiment, a plurality of Inputs (2601) may be passed to the network, and are passed through Encoding Blocks (2602). The Inputs (2601) may vary depending on the use of the Transformer Neural Network (2600). For example, it may be a standard Input Vector (1509) for Input Transformers (1802) or for a Single Transformer Neural Network (1701), but may be the concatenated vector of outputs (1804) for an output model (1805).

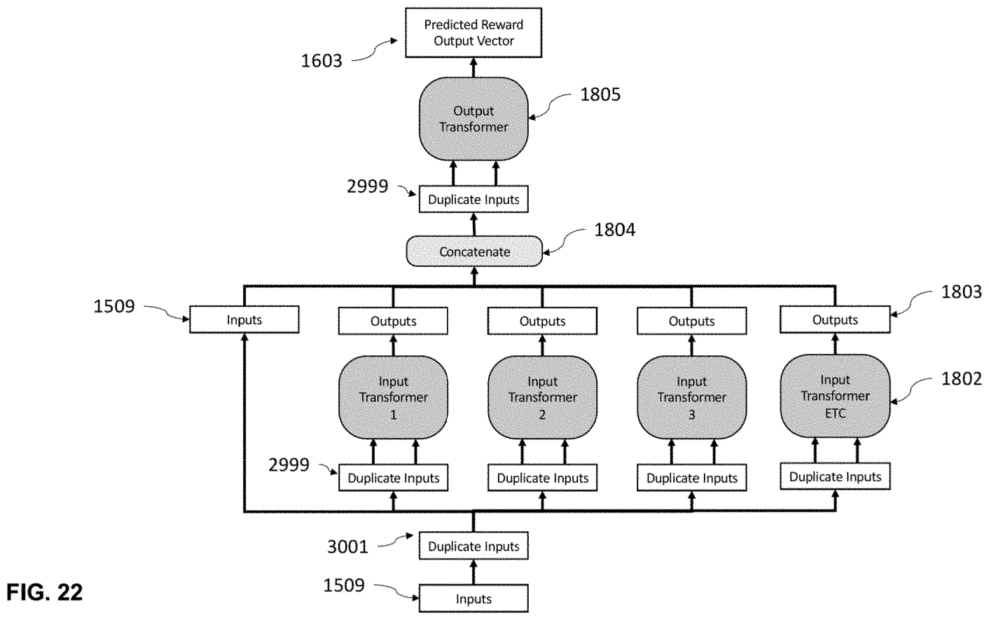

Element 1804 is given as an input in FIG. 17 in reference to the ensemble of Transformers Neural Network Component (1703). In FIG. 17, element (1804) is described as “consisting of both the Final Prepared Input Vector (1509) and concatenated Outputs (1803) from other transformer models”, meaning that it is an input to the final output Transformer Network, which consists of both the Full Input (1509) given to the Neural Network Component and the Outputs (1803) created by all of the various Input Transformers (1802). These are all combined together into one big new Input (1804) which is given to the Output Transformer (1805). This process is more clearly depicted in FIG. 22, which describes the distinction between the new Input 1804 and the Outputs 1803 included within the Input 1804.

In the same manner, Outputs (2605) may vary. For example, the Outputs (2605) may be the Predicted Reward Vector (1603), a Predicted Reward Vector for individual Metrics, a single numeric output of a prediction on a specific measure, a large latent-space vector, or other numeric values, which each offer various pros and cons. In one embodiment, there may be zero, one, or more Encoding Blocks, as demonstrated by “Nx” (2603) which demonstrates that this number may be changed to any amount. If the “Nx” (2603) for encoders is 0 (zero), the Inputs (2601) are given directly to a first Decoding Block (2604). The output of the final Encoder Block (2602) is then given to the first Decoder Block (2604) and is also given to each consecutive Decoder Block (2604). The output of the final Decoder Block (2604) is used as the Final Output (2605). If the “Nx” (2603) for decoders is 0, the output of the final Encoder Block (2602) is used as the Final Output (2605).

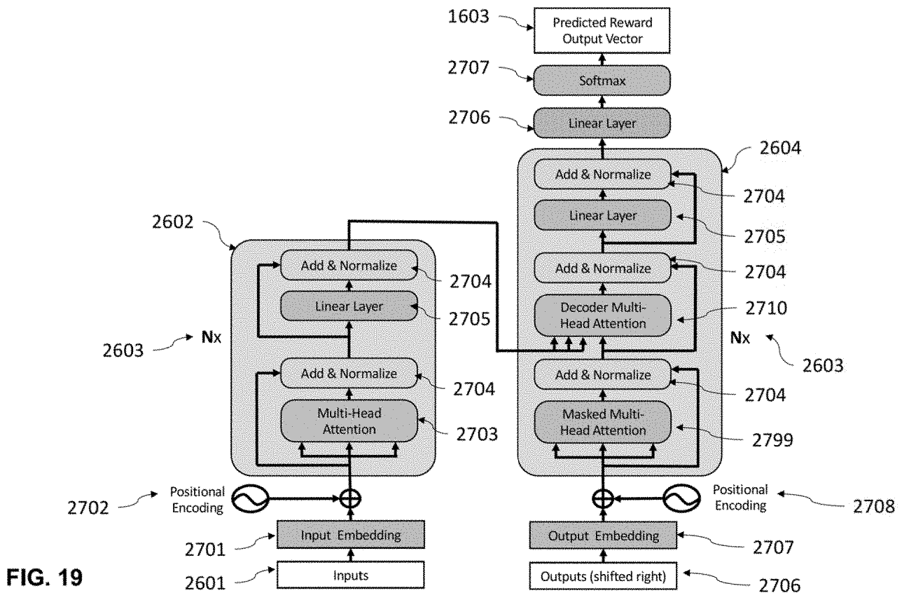

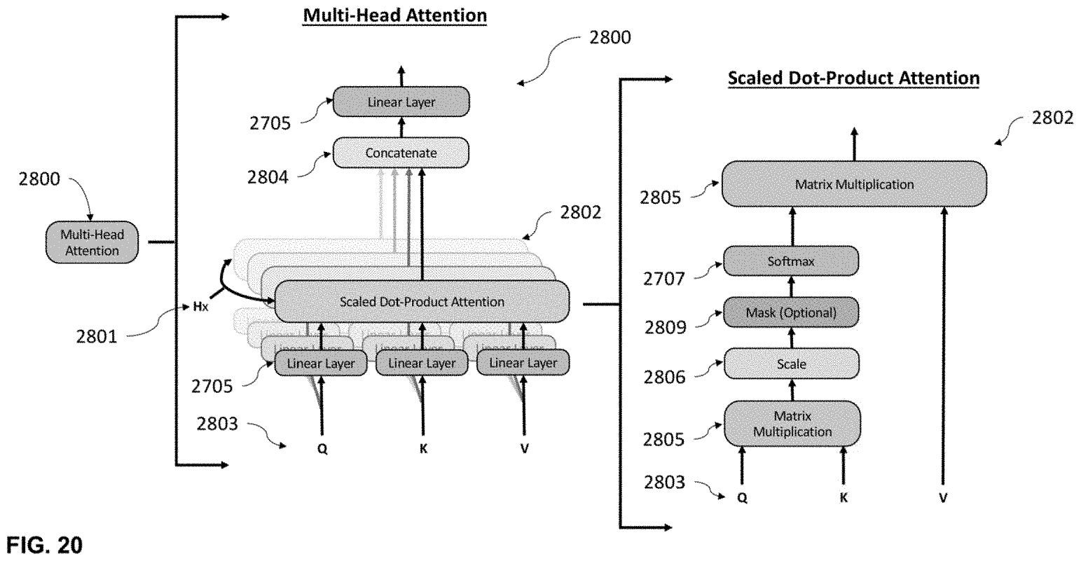

With respect to FIG. 19, a detailed flow diagram of additional componentry of the Transformer Neural Network (2600) of FIG. 18 is shown. In one embodiment, the Inputs (2601) are embedded in an Input Embedding component (2701) to store contextual information, then positionally encoded in a Positionally Encoded component (2702), and then passed to the first Encoder Block (2602). They Inputs (2601) may then be duplicated, one copy may be given to an Add & Normalize Layer (2704), and “Hx” (2801) copies are given to a Multi-Head Attention Layer (2703). In one embodiment, the Add & Normalize Layer (2704) may receive both the input copy and the output of the Multi-Head Attention Layer (2703), and the Add & Normalize Layer (2704) adds the input and output copies together, and normalizes the output which is then sent to both a Linear Layer (2705) and another Add & Normalize Layer (2704). The Linear Layer (2705) transmits its output to the next Add & Normalize Layer (2704), which adds the output with the same input received by the Linear Layer (2705), then normalizes the output and sends the output to the next encoder block. Once the inputs have passed through the “Nx” encoder blocks, the output of the final encoder block may be passed to all of the decoder blocks, and the original Inputs (2601) are shifted one timestep to the right (2706), given new embeddings (2707), i.e., a simple dense layer to map the Partially Designed Molecule (1507), Positionally Encoded with the positionally encoded component (2708), and given to the first Decoder Block (2604). The inputs to the Decoder Block (2604) may be duplicated, and one input may be passed to the Add & Normalize Layer (2704), and three copies (2803) are given to each of the “Hx” (2801) Masked Multi-Head Attention Layer (2799), which is the same as a Multi-Head Attention Layer (2703) except that it includes the optional Mask (2809) depicted in FIG. 20 below. An Add & Normalize Layer (2704) receives both the input copy and the output of the Masked Multi-Head Attention Layer (2799), adds them together, and normalizes the output which is then sent to the next Add & Normalize Layer (2704) and the Decoder Multi-Head Attention Layer (2710). The Decoder Multi-Head Attention Layer (2710) also receives the output from the final Encoder Block (2602), and then passes the processed output to the next Add & Normalize Layer (2704), which adds it with the original input received by the Decoder Multi-Head Attention Layer (2710). The Add & Normalize Layer (2704) normalizes the input and output, and passes it to both a Linear Layer (2705) and another Add & Normalize Layer (2704). The final Add & Normalize Layer (2704) sends this output to the next Decoder Block (2604), or if it is the final Decoder Block (2604), the output is sent to an output Linear Layer (2706), which, in one embodiment, may create the final Predicted Reward Output Vector (1603), and in another embodiment, may feed it into a Softmax Layer (2707) to create the final Predicted Reward Output Vector (1603). The SoftMax Layer (2707) is a common activation output layer used in Neural Networks which performs a SoftMax function, also known as a normalized exponential function. This function creates a normalized probability distribution over the predicted output classes. In some embodiments, it may not be needed for this model and so it may be an optional layer.

With respect to FIG. 20, a flow diagram of a Multi-Head Attention Layer component (2800) in one embodiment is shown on the left panel, and a corresponding flow diagram of a Scaled-Dot Product Attention component (2802) in one embodiment is shown on the right panel.

In one embodiment, the Multi-Head Attention Layer component (2800) receives three copies of inputs (2803) for each “Hx” (2801) number of attention heads, which are each passed through their own respective Linear Layers (2705) and given to the respective Scaled Dot-Product Attention Heads (2802). The outputs from all of the Scaled Dot-Product Attention Head (2802) are concatenated (2804), passed through another Linear Layer (2705) to create the final output of the Multi-Head Attention Layer (2800). On the right, a detailed flow diagram of the functions within the Scaled Dot-Product Attention Head component (2802) is shown. The Scaled Dot-Product Attention Head component (2802) receives three Input copies (2803), and performs Matrix Multiplication (2805) on two of the three Input copies (2803), and scales the Newly Multiplied Matrix with a Scales component (2806). The scaling is performed by dividing the new Matrix by the square root of the dimension of the Input Copies (2803). A Mask (2809) may optionally be applied next to make the layer a Masked Multi-Head Attention Layer (2799), which may provide for zeroing out numbers above the matrix diagonal. Next, a Softmax function is performed with a Softmax Layer (2707), and then sent alongside a remaining copy (2803) to another Matrix Multiplication Layer (2805) to create the final Scaled-Dot Product Attention output (2802).

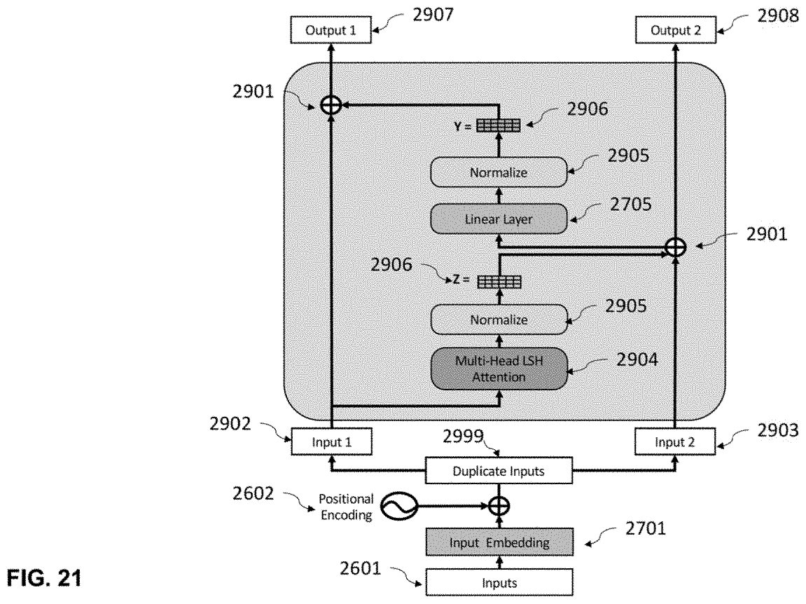

With respect to FIG. 21, a flow diagram of an Encoder Block (2602) with Reversible Residual Layers (2901) and Locality-Sensitive-Hashing (2902) is shown. In one embodiment, the present flow diagram process accomplishes similar tasks as the previous Encoder Blocks (2602). In one embodiment, Inputs (2601) are embedded in an embedding component (2701) to store contextual information, then positionally encoded in a Positionally Encoded component (2702), and then passed to the first Encoder Block (2602). The Inputs (2601) may be duplicated by a duplicator component (2999) into an Input 1 (2902) and an Input 2 (2903) and used for two identical copies of the model. In the first model copy, Input 1 (2902) is passed to a Multi-Head LSH Attention Layer (2904). The term LSH stands for “locality-sensitive hashing”, which is very similar to the previous Multi-Head Attention Layers (2800) except that the Multi-Head LSH Attention Layer (2904) uses locality-sensitive hashing (LSH) rather than full dot-product matrix multiplication. The Multi-Head LSH Attention Layer (2904) output is then passed to a Normalization Layer (2905) to create an output Z (2906). The Output Z (2906) may then be used as an Output 2 (2908) which is one of the two model outputs, but in the second model copy Output 2 (2908) may be added to Input 2 (2903) then passed to a Linear Layer (2705). The Linear Layer (2705) passes its output to another Normalization Layer (2905) to create an Output Y (2907), which is added to a copy of Input 1 (2902) and used as Output 1 (2907), which is the other model output. Splitting the Add & Normalization Layers (2704) into computing the addition section separately in different model copies allows activations to be recalculated during backpropagation so that the different model copies do not have to be stored, dramatically reducing memory requirements. These same concepts of LSH and Reversible Residual Layers can be applied to Decoder Blocks in the same way, and provide a significantly computationally efficient implementation of the Transformer Neural Network (2600) in some scenarios.

With respect to FIG. 22, a flow diagram of a Multiple-Transformer Neural Network (1703) is shown. In one embodiment, the Inputs (1509) described above may be duplicated (3001), once for every Input Transformer (1802) used and another one to be concatenated with an Input Transformers' Outputs (1803). There may be any number of one or more Input Transformers (1802) used, thus depicted herein is the use of four Input Transformers (1802) each sequentially numbered, with the fourth Input Transformers (1802) given an “ETC” in the place of a number to display the use any number of identical transformers. All of the Input Transformers (1802) create their own respective Outputs (1803), which may be a Predicted Reward Vector (1603) for individual Metrics, a single numeric output of a prediction on a specific measure, a large latent-space vector, or other numeric values. The remaining copy of Inputs (1509) and all of the Outputs (1803) may be concatenated together with a concatenating component (1804) into a single input vector which may be duplicated and passed to the Output Transformer (1805). The Output Transformer (1805) may create the Predicted Reward Output Vector (1603) which may then be used as the final output for the One or More Neural Network Component (1602) (see FIG. 16).

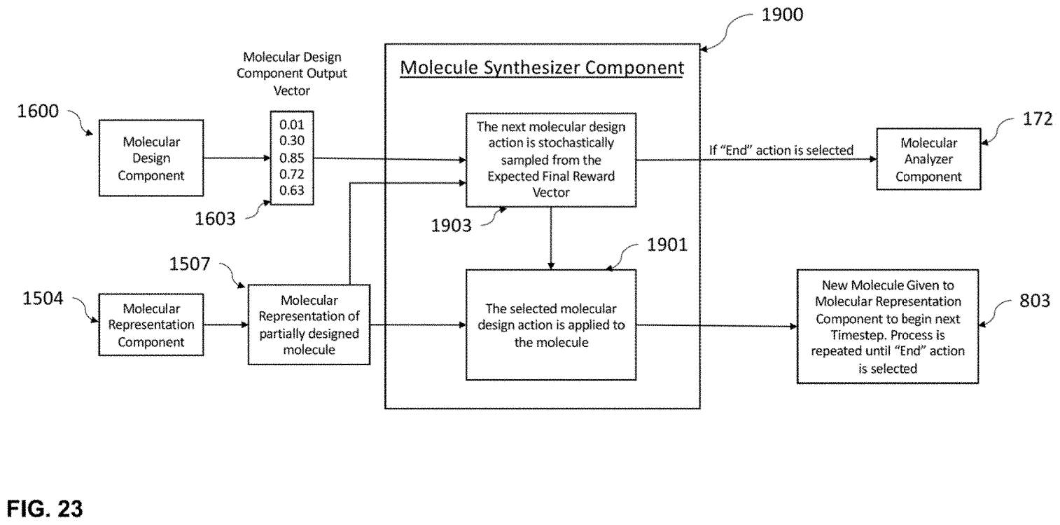

With respect to FIG. 23, a flow diagram of the Molecule Synthesizer Component (1900) functionality is shown. The Molecule Synthesizer Component (1900) simultaneously receives both the Molecular Design Component Output Vector (1603) and the Molecular Representation of the Partially Designed Molecule (1507) as inputs. Initially, before any processing of a molecule, the Partially Designed Molecule (1507) can be a matrix or other numeric representation where all values are set as 0, in other words an initial Partially Designed Molecule (1507) having a 0 (zero) for all values. Alternatively, a known molecule having known attributes, or unknown attributes, may be used as the initial partially designed molecule (1507). First, the Molecule Synthesizer Component (1900) uses stochastic sampling (to introduce variation) to select the next Molecular Design Action (1903) from the Molecular Design Component Output Vector (1603). If the selected action is not the “End” action, the Molecule Synthesizer Component (1900) updates the Molecular Representation of the Partially Designed Molecule (1507) to reflect the selected molecular design action being synthesized. For example, in one embodiment, this update may include concatenating the previously used Numerical Matrix Representation (1505) with another vector of individual pieces of molecular information such as atom types and coordinates, molecular bonds, or other molecular properties to create a new, updated Numerical Matrix Representation (1505). This updated Numerical Matrix Representation (1505) is received by the Molecular Representation Component (1504) to begin the next step of molecular design as depicted in FIG. 8 (intermediary input matrix 804). Alternatively, if the next molecular design action selected by the Molecule Synthesizer Component (1900) is the “End” action, the molecular representation of the partially designed molecule becomes the Final New Molecule (1801) and is received by the Molecular Analyzer Component (172).

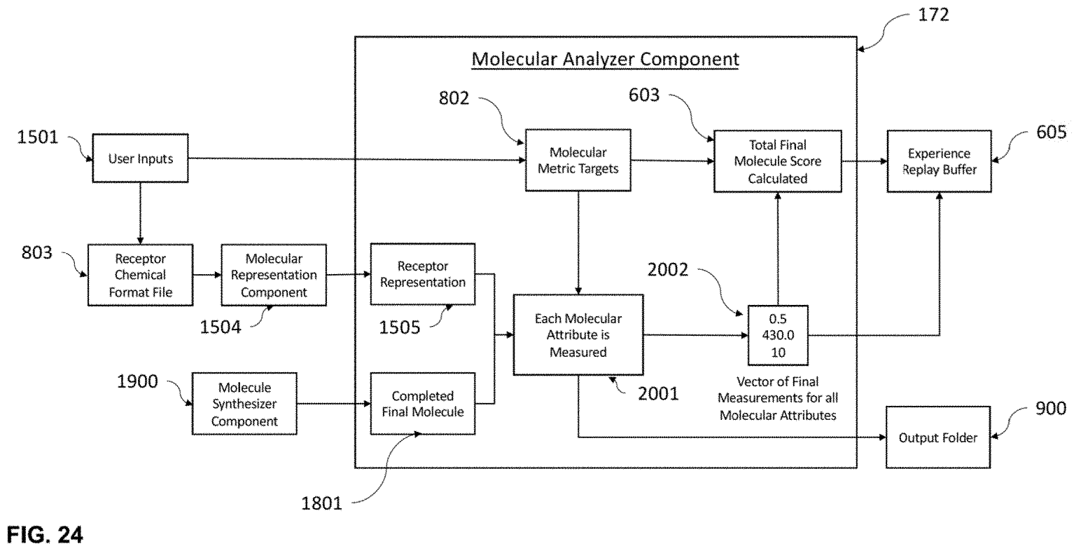

With respect to FIG. 24, a flow diagram of the Molecular Analyzer Component (172) is shown. As inputs, the Molecular Analyzer Component (172) receives the Molecule Metric Targets (802) from the User Inputs, all (if any) Receptor Representations used to calculate the target molecular metric scores from the Molecular Representation Component (1504), and the Final New Molecule (1801) from the Molecule Synthesizer Component (1900). These inputs are used to measure each molecular attribute of the Final New Molecule (1801), and all molecular measurement output files are saved to the Output Folder (900). In various embodiments, each molecular attribute may be measured using at least one or more of the following molecular measurement tools: Neural Networks as depicted in FIG. 18 (1805), other forms of Machine Learning, third-party software (e.g. RDKit or AutoDock Vina), or custom metric calculation functions.

A vector representation of all Final Molecular Measurement Scores (2002) of the Final New Molecule (1801) may then be created and each molecular measurement score may be saved to its respective Experience Replay Buffer (605). This vector and the Molecular Metric Targets are then used to compute the Total Final Molecule Score (603). In one embodiment, this may be calculated by assigning importance scores of 0 to each molecular metric not selected as a molecular metric goal, multiplying the importance scores by the respective Final Molecular Measurement Scores (2002), and taking the sum of all of these products. The Total Final Molecule Score (603) is then saved to the primary Experience Replay Buffer (605) which holds the training data for the final (or only) output Neural Network.

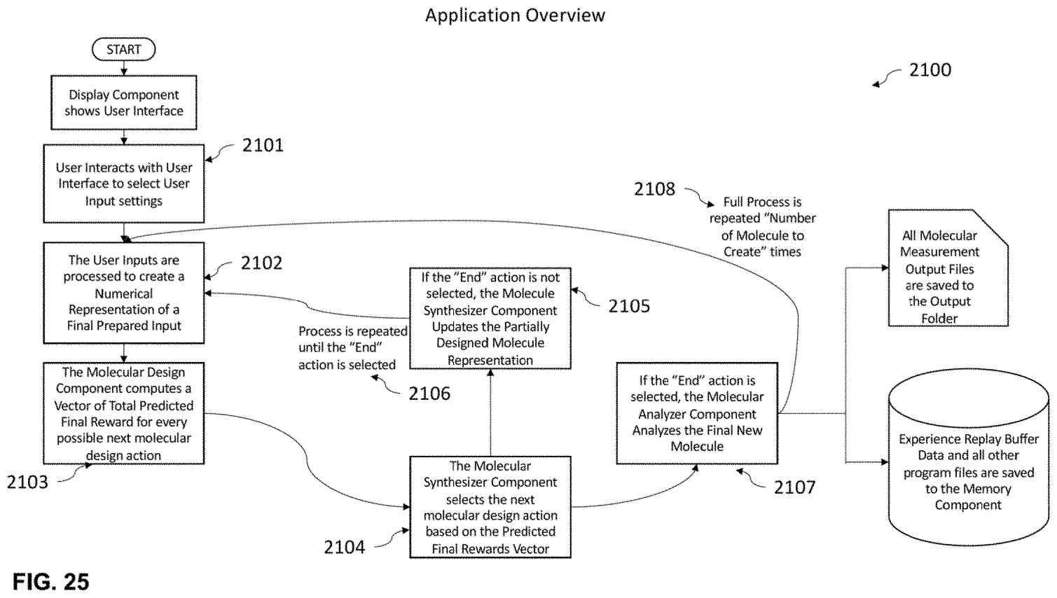

With respect to FIG. 25, a flow diagram of an overview (2100) for a system for automated targeted intentional molecular design is shown. At a step (2101), a user uses the computer keyboard and mouse to input user settings and begin the molecule design process. At a step (2102), all User Input settings are processed into a numerical matrix representation (1509). At a step (2103), a Molecular Design Component computes a Vector of Total Predicted Final Reward (1603) for every possible next molecular design action. At a step (2104), a Molecule Synthesizer Component (1900) selects a next action in the molecular design process. At a step (2105), a Molecule Synthesizer Component (1900) updates the numerical matrix representation of a partially designed molecule to complete the next molecular design action. At a step (2106), steps (2102, 2103, 2104, 2105) are repeated until the “End” action is selected by the Molecule Synthesizer Component (1900). At a step (2107), a Molecular Analyzer Component (172) receives a Final New Molecule from the Molecule Synthesizer Component (1900), analyzes the Molecule Synthesizer Component (1900), measuring a wide variety of its molecular attributes, saves all molecule metric output files to the Output Folder (900), and saves all used for the Experience Replay Buffer (605) to the Memory Component (127). At a step (2108), steps (2102, 2103, 2104, 2105, 2106, 2107) are repeated until the “Number of Molecules to Create” defined by the User Inputs (1501) have been completed.

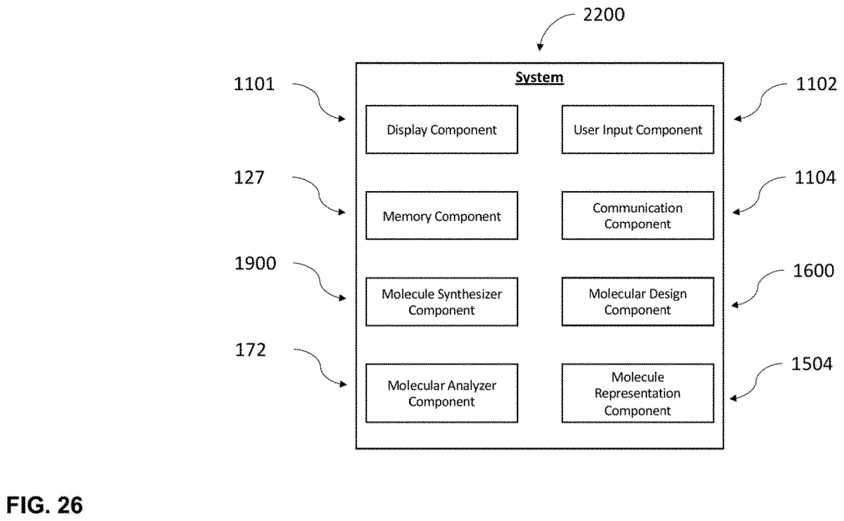

With respect to FIG. 26, a block diagram of the system 2200 for automated targeted intentional molecular design is shown. The system (2200) may include a Display Component (1101), a User Input Component (1102), a Memory Component (1103), a Communication Component (1104), a Molecule Synthesizer Component (1900), a Molecular Design Component (1600), a Molecule Analyzer Component (172), and a Molecule Representation Component (1504). In one embodiment, the Display Component (1101) displays the User Interface on the System (2200), which the user may interact with using the User Input Component (1102). In one embodiment, the User Input Component (1102) may consist of a keyboard and/or mouse, a touchscreen in another embodiment, or other input devices in other embodiments. The Memory Component (127) may contain protein receptor files, Experience Replay Buffer (605) data, previous experiment history, and other past files uploaded by the user.

The Communication Component (1104) may be configured to establish a connection between the System (2200) and any number of external molecule databases in order to send and/or retrieve additional molecule data for the Memory Component (127). The Molecule Synthesizer Component (1900) may be configured to select top-scoring molecular design actions based on the Predicted Final Score Output Vector (1603) provided by the Molecular Design Component (1600) The Molecular Design Component (1600) may consist of one or many Neural Networks (1602) used to predict a Vector of Total Predicted Final Reward (1603), given to a final molecule by the Molecular Analyzer Component (172), for every possible next molecular design action which may be selected by the Molecule Synthesizer Component (1900). The Molecule Analyzer Component (172) may be configured to assign measurements and/or scores to newly designed molecules, for a large variety of molecular attributes. The Molecule Representation Component (1504) may be configured to convert the representations of molecules between different molecular representation including but not limited to SMILE format representation, binary array representation, 3-D structural graph representation, and any other molecular representation format needed by other components within the System (2200).

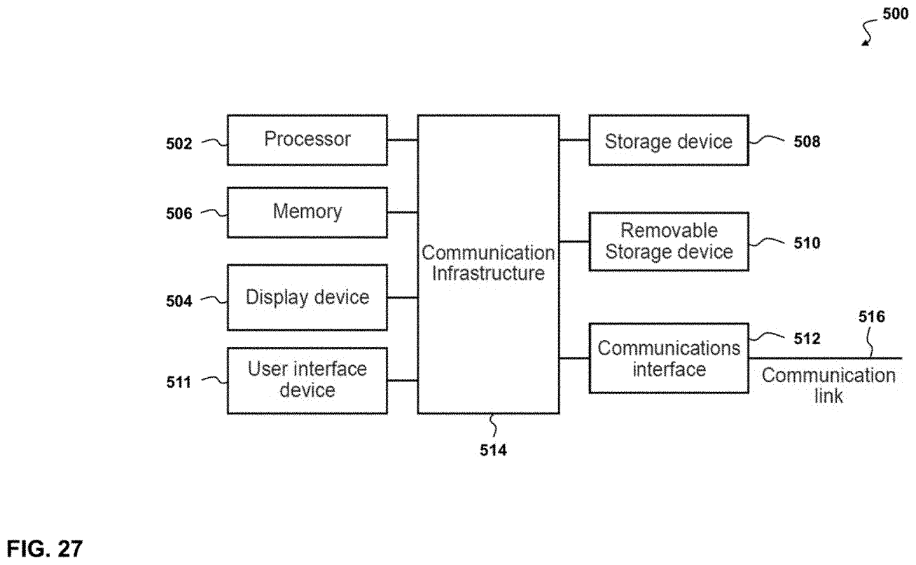

FIG. 27 is a high-level block diagram (500) showing a computing system comprising a computer system useful for implementing an embodiment of the system and process, disclosed herein. Embodiments of the system may be implemented in different computing environments. The computer system includes one or more processors (502), and can further include an electronic display device (504) (e.g., for displaying graphics, text, and other data), a main memory (506) (e.g., random access memory (RAM)), storage device (508), a removable storage device (510) (e.g., removable storage drive, Graphics Processing Unit (GPU), a removable memory module, a magnetic tape drive, an optical disk drive, a computer readable medium having stored therein computer software and/or data), user interface device (511) (e.g., keyboard, touch screen, keypad, pointing device), and a communication interface (512) (e.g., modem, a network interface (such as an Ethernet card), a communications port, or a PCMCIA slot and card). The communication interface (512) allows software and data to be transferred between the computer system and external devices. The system further includes a communications infrastructure (514) (e.g., a communications bus, cross-over bar, or network) to which the aforementioned devices/modules are connected as shown.

Information transferred via communications interface (514) may be in the form of signals such as electronic, electromagnetic, optical, or other signals capable of being received by communications interface (514), via a communication link (516) that carries signals and may be implemented using wire or cable, fiber optics, a phone line, a cellular/mobile phone link, a radio frequency (RF) link, and/or other communication channels. Computer program instructions representing the block diagram and/or flowcharts herein may be loaded onto a computer, programmable data processing apparatus, or processing devices to cause a series of operations performed thereon to produce a computer-implemented process.

Embodiments have been described with reference to flowchart illustrations and/or block diagrams of methods, apparatus (systems) and computer program products according to embodiments. Each block of such illustrations/diagrams, or combinations thereof, can be implemented by computer program instructions. The computer program instructions when provided to a processor produce a machine, such that the instructions, which execute via the processor, create means for implementing the functions/operations specified in the flowchart and/or block diagram. Each block in the flowchart/block diagrams may represent a hardware and/or software module or logic, implementing embodiments. In alternative implementations, the functions noted in the blocks may occur out of the order noted in the figures, concurrently, etc.

Computer programs (i.e., computer control logic) are stored in main memory and/or secondary memory. Computer programs may also be received via a communications interface (512). Such computer programs, when executed, enable the computer system to perform the features of the embodiments as discussed herein. In particular, the computer programs, when executed, enable the processor and/or multi-core processor to perform the features of the computer system. Such computer programs represent controllers of the computer system.

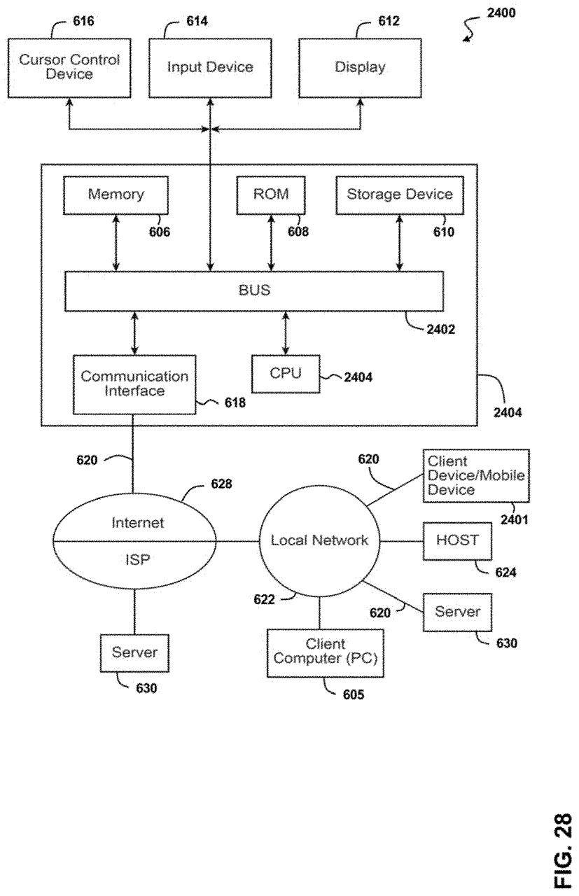

FIG. 28 shows a block diagram of an example system (2400) in which an embodiment may be implemented. The system (2400) includes one or more client devices (2401) such as consumer electronics devices, connected to one or more server computing systems (630). A server (630) includes a bus (2402) or other communication mechanism for communicating information, and a processor (CPU and/or GPU) (2404) coupled with the bus (2402) for processing information. The server (630) also includes a main memory (606), such as a random-access memory (RAM) or other dynamic storage device, coupled to the bus (2402) for storing information and instructions to be executed by the processor (2404). The main memory (606) also may be used for storing temporary variables or other intermediate information during execution or instructions to be executed by the processor (2404). The server computer system (630) further includes a read only memory (ROM) (608) or other static storage device coupled to the bus (2402) for storing static information and instructions for the processor (2404). A storage device (610), such as a magnetic disk or optical disk, is provided and coupled to the bus (2402) for storing information and instructions. The bus (2402) may contain, for example, thirty-two address lines for addressing video memory or main memory (606). The bus (2402) can also include, for example, a 32-bit data bus for transferring data between and among the components, such as the CPU (2404), the main memory (606), video memory and the storage (610). Alternatively, multiplex data/address lines may be used instead of separate data and address lines.

The server (630) may be coupled via the bus (2402) to a display (612) for displaying information to a computer user. An input device (614), including alphanumeric and other keys, is coupled to the bus (2402) for communicating information and command selections to the processor (2404). Another type or user input device comprises cursor control (616), such as a mouse, a trackball, or cursor direction keys for communicating direction information and command selections to the processor (2404) and for controlling cursor movement on the display (612).

According to one embodiment, the functions are performed by the processor (2404) executing one or more sequences of one or more instructions contained in the main memory (606). Such instructions may be read into the main memory (606) from another computer-readable medium, such as the storage device (610). Execution of the sequences of instructions contained in the main memory (606) causes the processor (2404) to perform the process steps described herein. One or more processors in a multi-processing arrangement may also be employed to execute the sequences of instructions contained in the main memory (606). In alternative embodiments, hard-wired circuitry may be used in place of or in combination with software instructions to implement the embodiments. Thus, embodiments are not limited to any specific combination of hardware circuitry and software.

The terms “computer program medium,” “computer usable medium,” “computer readable medium”, and “computer program product,” are used to generally refer to media such as main memory, secondary memory, removable storage drive, a hard disk installed in hard disk drive, and signals. These computer program products are means for providing software to the computer system. The computer readable medium allows the computer system to read data, instructions, messages or message packets, and other computer readable information from the computer readable medium. The computer readable medium, for example, may include non-volatile memory, such as a floppy disk, ROM, flash memory, disk drive memory, a CD-ROM, and other permanent storage. It is useful, for example, for transporting information, such as data and computer instructions, between computer systems. Furthermore, the computer readable medium may comprise computer readable information in a transitory state medium such as a network link and/or a network interface, including a wired network or a wireless network that allow a computer to read such computer readable information. Computer programs (also called computer control logic) are stored in main memory and/or secondary memory. Computer programs may also be received via a communications interface. Such computer programs, when executed, enable the computer system to perform the features of the embodiments as discussed herein. In particular, the computer programs, when executed, enable the processor multi-core processor to perform the features of the computer system. Accordingly, such computer programs represent controllers of the computer system.

Generally, the term “computer-readable medium” as used herein refers to any medium that participated in providing instructions to the processor (2404) for execution. Such a medium may take many forms, including but not limited to, non-volatile media, volatile media, and transmission media. Non-volatile media includes, for example, optical or magnetic disks, such as the storage device (610). Volatile media includes dynamic memory, such as the main memory (606). Transmission media includes coaxial cables, copper wire and fiber optics, including the wires that comprise the bus (2402). Transmission media can also take the form of acoustic or light waves, such as those generated during radio wave and infrared data communications.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, an EPROM, a FLASH-EPROM, any other memory chip or cartridge, a carrier wave as described hereinafter, or any other medium from which a computer can read.

Various forms of computer readable media may be involved in carrying one or more sequences of one or more instructions to the processor (2404) for execution. For example, the instructions may initially be carried on a magnetic disk of a remote computer. The remote computer can load the instructions into its dynamic memory and send the instructions over a telephone line using a modem. A modem local to the server (630) can receive the data on the telephone line and use an infrared transmitter to convert the data to an infrared signal. An infrared detector coupled to the bus (2402) can receive the data carried in the infrared signal and place the data on the bus (2402). The bus (2402) carries the data to the main memory (606), from which the processor (2404) retrieves and executes the instructions. The instructions received from the main memory (606) may optionally be stored on the storage device (610) either before or after execution by the processor (2404).

The server (630) also includes a communication interface (618) coupled to the bus (2402). The communication interface (618) provides a two-way data communication coupling to a network link (620) that is connected to the worldwide packet data communication network now commonly referred to as the Internet (628). The Internet (628) uses electrical, electromagnetic or optical signals that carry digital data streams. The signals through the various networks and the signals on the network link (620) and through the communication interface (618), which carry the digital data to and from the server (630), are exemplary forms or carrier waves transporting the information.

In another embodiment of the server 630, interface 618 is connected to a network 622 via a communication link 620. For example, the communication interface 618 may be an integrated services digital network (ISDN) card or a modem to provide a data communication connection to a corresponding type of telephone line, which can comprise part of the network link 620. As another example, the communication interface 618 may be a local area network (LAN) card to provide a data communication connection to a compatible LAN. Wireless links may also be implemented. In any such implementation, the communication interface 618 sends and receives electrical electromagnetic or optical signals that carry digital data streams representing various types of information.

The network link 620 typically provides data communication through one or more networks to other data devices. For example, the network link 620 may provide a connection through the local network 622 to a host computer 624 or to data equipment operated by an Internet Service Provider (ISP). The ISP in turn provides data communication services through the Internet 628. The local network 622 and the Internet 628 both use electrical, electromagnetic or optical signals that carry digital data streams. The signals through the various networks and the signals on the network link 620 and through the communication interface 618, which carry the digital data to and from the server 630, are exemplary forms or carrier waves transporting the information.

The server 630 can send/receive messages and data, including e-mail, program code, through the network, the network link 620 and the communication interface 618. Further, the communication interface 618 can comprise a USB/Tuner and the network link 620 may be an antenna or cable for connecting the server 630 to a cable provider, satellite provider or other terrestrial transmission system for receiving messages, data and program code from another source.

The example versions of the embodiments described herein may be implemented as logical operations in a distributed processing system such as the system 2400 including the servers 630. The logical operations of the embodiments may be implemented as a sequence of steps executing in the server 630, and as interconnected machine modules within the system 2400. The implementation is a matter of choice and can depend on performance of the system 2400 implementing the embodiments. As such, the logical operations constituting said example versions of the embodiments are referred to for e.g., as operations, steps or modules.

Similar to a server 630 described above, a client device 2401 can include a processor, memory, storage device, display, input device and communication interface (e.g., e-mail interface) for connecting the client device to the Internet 628, the ISP, or LAN 622, for communication with the servers 630.

The system 2400 can further include computers (e.g., personal computers, computing nodes) 605 operating in the same manner as client devices 2401, where a user can utilize one or more computers 605 to manage data in the server 630.

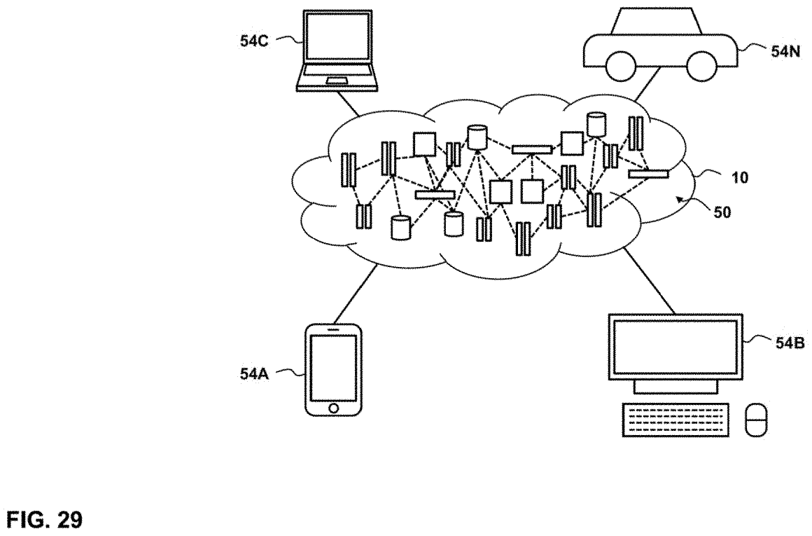

Referring now to FIG. 29, illustrative cloud computing environment 50 is depicted. As shown, cloud computing environment 50 comprises one or more cloud computing nodes 10 with which local computing devices used by cloud consumers, such as, for example, personal digital assistant (PDA), smartphone, smart watch, set-top box, video game system, tablet, mobile computing device, or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 25 are intended to be illustrative only and that computing nodes 10 and cloud computing environment 50 can communicate with any type of computerized device over any type of network and/or network addressable connection (e.g., using a web browser).

It is contemplated that various combinations and/or sub-combinations of the specific features and aspects of the above embodiments may be made and still fall within the scope of the invention. Accordingly, it should be understood that various features and aspects of the disclosed embodiments may be combined with or substituted for one another in order to form varying modes of the disclosed invention. Further, it is intended that the scope of the present invention is herein disclosed by way of examples and should not be limited by the particular disclosed embodiments described above.

What is claimed is:

1. A method of generating at least one of the chemical and physical structure of at least one molecule having a property, comprising:
providing an initial molecule having at least one of a chemical structure and a physical structure;
selecting at least a first attribute of the initial molecule relating to a first property thereof;
evaluating the performance of the first molecule with respect to the first property thereof;
modifying at least a portion of the at least one of a chemical structure and a physical structure of the initial molecule to form a first modified molecule;
predicting the performance of the first modified molecule, upon further modification thereof, with respect to the performance of that first modified molecule with respect to the first property thereof; and
based on the predicted performance, further modifying the first modified molecule.

2. The method of claim 1, further comprising:
modifying at least a portion of the at least one of a chemical structure and a physical structure of the initial molecule to form second through nth modified molecules, where n is a positive integer; and
predicting the performance of the second through n−1 modified molecules, upon further modification thereof, with respect to the performance of that second through n−1 modified molecules with respect to the property thereof, and
based on the predicted performance, further modify each of the first through n−1 modified molecules to generate the nth modified molecule.

3. The method of claim 2, wherein the performance of each of the second through n−1 modified molecules, upon further modification thereof, is predicted before a next molecule of the second to n−1 molecules is generated.

4. The method of claim 2, wherein at least two different changes to the at least one of a chemical structure and a physical structure are made to the same previously modified molecule to create two candidate molecules, before the performance of the at least two candidate molecules with respect to the property thereof upon further modification thereof, is predicted.

5. The method of claim 4, wherein, as among the at least two candidate molecules, the one with the best predicted performance with respect to the property thereof, is modified to form the next one of the second through n−1 molecules.

6. The method of claim 1, wherein the property thereof is binding energy.

7. The method of claim 1, wherein the property thereof is the location of a potential chemical binding site with respect to the topography of the nth molecule.

8. The method of claim 1, further comprising;
selecting a second attribute of the initial molecule relating to a second property thereof;
evaluating the performance of the molecule with respect to the first and the second property thereof;
modifying at least a portion of the at least one of a chemical structure and a physical structure of the initial molecule to form a first modified molecule;
predicting the performance of the first modified molecule, upon further modification thereof, with respect to the performance of that first modified molecule with respect to the first and the second property thereof.

9. The method of claim 8, further comprising:
selecting a third attribute of the initial molecule relating to a third property thereof;
evaluating the performance of the molecule with respect to the first, the second and the property thereof;
modifying at least a portion of the at least one of a chemical structure and a physical structure of the initial molecule to form a first modified molecule;
predicting the performance of the first modified molecule, upon further modification thereof, with respect to the performance of that first modified molecule with respect to the first, the second and the third property thereof.

10. The method of claim 1, further comprising:
providing a second through an mth initial molecule, the second through mth initial molecules having at least one of a chemical structure and a physical structure;
selecting at least a first attribute of each of the second through mth initial molecules relating to a first property thereof;
evaluating the performance of each of the second through mth initial molecules with respect to the first property thereof;
modifying at least a portion of the at least one of a chemical structure and a physical structure of the of each of the second through nth initial molecules to form a first modified second through nth molecule;
predicting the performance of the first modified second through nth molecule, upon further modification thereof, with respect to the performance of that first modified molecule with respect to the first property thereof.

11. The method of claim 10, further comprising, for each of the second through nth initial molecules:
modifying at least a portion of the at least one of a chemical structure and a physical structure of each of the second through nth initial molecules to form second through nth modified second through nth molecules, where n is a positive integer; and
predicting the performance of the second through n−1 modified molecules, upon further modification thereof, with respect to the performance of that second through n−1 modified molecules with respect to the property thereof.

12. The method of claim 11, further comprising ranking the performance of each of the first through nth molecules with respect to the property thereof.

13. A non-transitory computer-readable medium comprising instructions that, when executed by one or more processors of a computing system, cause the computing system to iteratively generate one or more molecular structures having desirable molecule properties comprising;
representing user inputs in the form of a numeric matrix of one or more dimensions;
predicting, using a model, a final metric or score assigned to a generated molecule upon completion for one or more actions, if that action were to be used as the next design action taken in the generation of one or more molecules;
selecting one or more actions based on the predicted metric or scores; and
generating one or more molecules based upon the selected actions.

14. The non-transitory computer-readable medium of claim 13, the instructions further comprising:
generating an initial numeric matrix representative of a molecule structure received from a user input.

15. The non-transitory computer-readable medium of claim 14, the instructions further comprising:
after predicting, using a model, a final metric or score assigned to a generated molecule upon completion for one or more actions, if that action were to be used as the next design action taken in the generation of one or more molecules and selecting one or more actions based on the predicted metric or scores and generating a molecule based on the selected actions a first time, repeating predicting, using a model, a final metric or score assigned to a generated molecule upon completion for one or more actions, if that action were to be used as the next design action taken in the generation of one or more molecules and selecting one or more actions based on the predicted metric or scores and generating a molecule based on the selected actions n additional times, where n is a positive, whole number integer.

16. The non-transitory computer readable medium of claim 15, further comprising selecting n based on a user input to the non-transitory computer readable medium.

17. The non-transitory computer-readable medium of claim 13, wherein the one or more dimensions include an initial molecule represented in SMILE format.

18. The non-transitory computer-readable medium of claim 13, wherein the one or more dimensions include an initial molecule represented in chemical file format.

19. The non-transitory computer-readable medium of claim 13, further comprising a table generator to tabulate the properties of one or more molecules generated by the computer readable media.

20. The non-transitory computer-readable medium of claim 13, wherein selecting one or more actions based on the predicted metric or scores and generating a molecule based on the selected actions includes accessing relative importance weights for different molecular properties and using the relative importance weights to predict metric or scores and generate a molecule based on the selected actions.
